(19) United States
(12) Patent Application Publication
(10) Pub. No.: US 2024/0263220 A1
(43) Pub. Date: Aug. 8, 2024

(54) IN SITU ANALYSIS OF VARIANT SEQUENCES IN BIOLOGICAL SAMPLES

(71) Applicant: 10x Genomics, Inc., Pleasanton, CA (US)

(72) Inventor: Mimmi OLOFSSON, Oakland, CA (US)

(21) Appl. No.: 18/431,779

(22) Filed: Feb. 2, 2024

Related U.S. Application Data
(60) Provisional application No. 63/443,283, filed on Feb. 3, 2023.

(51) Int. Cl. C12Q 1/6841; C12Q 1/682; C12Q 1/6827

(52) U.S. Cl. CPC C12Q 1/6841; C12Q 1/682; C12Q 1/6827; C12Q 2600/156; C12Q 2600/16

(57) ABSTRACT

The present disclosure relates in some aspects to methods for analyzing target nucleic acids in a biological sample. In some aspects, the presence, amount, and/or identity of a plurality of different target nucleic acids and/or variant sequences (e.g., SNPs or mutations) of one or more of the different target nucleic acids are analyzed in situ in a sample. Also provided are oligonucleotides, sets of oligonucleotides, compositions, and kits for use in accordance with the methods.

### CROSS-REFERENCE TO RELATED APPLICATIONS

This application claims priority to U.S. Provisional Patent Application No. U.S. 63/443,283 filed Feb. 3, 2023, entitled “IN SITU ANALYSIS OF VARIANT SEQUENCES IN BIOLOGICAL SAMPLES,” which is herein incorporated by reference in its entirety for all purposes.

### FIELD

The present disclosure relates in some aspects to methods and compositions for nucleic acid analysis in situ in biological samples, such as multiplex genotyping of single nucleotide differences in target nucleic acid molecules in situ in a cell or tissue sample.

### BACKGROUND

Methods are available for detecting nucleic acids present in a biological sample. For instance, advances in single molecule fluorescent in situ hybridization (smFISH) have enabled nanoscale-resolution imaging of RNA in cells and tissues. However, analysis of short sequences (e.g., single nucleotide differences such as single nucleotide polymorphisms (SNPs) or point mutations) on individual transcripts in situ has remained challenging. Improved methods for identifying these variant sequences and analyzing their spatial distribution in cell or tissue samples are needed. Provided herein are methods, compositions, and kits that address such and other needs.

### BRIEF SUMMARY

Certain assays using probe hybridization/ligation to discriminate variant sequences can suffer from low specificity for multiple reasons, including properties of the ligase and/or the target nucleic acid. Low ligase fidelity can result in formation and detection of a ligation product (and subsequent amplicons of the ligation product), even when a sequence in a nucleic acid molecule does not match the interrogatory region (e.g., interrogatory hybridization region) of a probe, producing a high level of background or false positive results. For example, the interrogatory region of the probe can hybridize to a sequence that differs from the sequence of interest (which matches the interrogatory region of the probe) by one or more nucleotide residues, and the mismatched probe can lead to incorrect ligation products since RNA templated ligases can tolerate some mismatches. In addition, ligases can have a strong base preference and probe end bias. In the case of circularizable probes targeting a single nucleotide difference, the single nucleotide is usually targeted by one arm, while the other arm covers a common region (e.g., a conserved or constant region) among nucleic acid molecules containing different bases at the single nucleotide position (e.g., probe for detecting SNP 1 as shown in FIG. 1A). For instance, one arm of an incorrect circularizable probe (e.g., probe for detecting SNP 2 as shown in FIG. 1B) could hybridize to the common region in the target nucleic acid molecule, whereas the other arm of the circularizable probe does not fully match the single nucleotide position in the target nucleic acid molecule (e.g., as shown in FIG. 1B, the probe for SNP 2 incorrectly hybridizes to the target nucleic acid comprising SNP 1), leading to an incorrect ligation product (e.g., a circularized probe corresponding to SNP 2 generated on a transcript containing SNP 1). In other instances, one arm of a correct circularizable probe (e.g., probe for detecting SNP 1 as shown in FIG. 1C) could perfectly match the single nucleotide position in the target nucleic acid molecule, but the other arm of the correct circularizable probe cannot hybridize to the common region which is occupied by an incorrect circularizable probe or another probe (e.g., as shown in FIG. 1C, the common region of the target nucleic acid is occupied by the probe for SNP 2). This is either ligated to a two-probe chimera that cannot be amplified by rolling circle amplification (RCA), or probe hybridization can become unstable so that neither of the circularizable probes generates a ligation product and subsequently a detectable RCA product (RCP), which can lead to a drop in detection efficiency. Improved methods for analyzing nucleic acids present in a biological sample, such as for in situ SNP genotyping, are needed.

In some embodiments, provided herein are methods comprising contacting target nucleic acids with a partner probe (e.g., a constant probe) and an interrogatory probe (e.g., for detecting a variation, e.g., an SNV/SNP-specific probe) which, upon target hybridization, can be ligated to the partner probe and form a circularized probe. In some embodiments, multiple interrogatory probes are provided as separate molecules from the partner probe that is non-interrogatory. For instance, as shown in FIG. 1D, the partner probe can comprise a stable binding arm that hybridizes to a conserved sequence outside one or more nucleotide(s) of interest (variable target sequence e.g., SNP 1, SNP 2, and SNP 3 as shown in the figure) or point mutations to be detected, whereas multiple interrogatory probes can compete with each other for hybridization to a particular SNP or point mutation. In some embodiments, an interrogatory hybridization region of each interrogatory probe comprises a sequence that is complementary to the variable target sequence (e.g., a region comprising one or more nucleotides of interest, such as SNPs or mutation hotspots) in a target nucleic acid in the biological sample. Thus, interrogatory probes comprising different interrogatory hybridization regions can compete for hybridization to a target nucleic acid molecule, allowing the best matching interrogatory probe to outcompete other interrogatory probes without interference from a non-interrogatory region that may decrease probe hybridization efficiency of the interrogatory hybridization region. Having an interrogatory hybridization region (e.g., in an interrogatory probe) and a non-interrogatory region (e.g., a constant hybridization region in a partner probe) in physically separate molecules until they are ligated to each other may allow more efficient and effective competition among various interrogatory hybridization regions, increase hybridization specificity, and/or help reduce or avoid the probe competition problems associated with probes that are hybridized to target molecules as one piece.

In some embodiments, disclosed herein is a method for analyzing a biological sample, the method comprising contacting the biological sample with a partner probe and an interrogatory probe for a first target nucleic acid. In some embodiments, the partner probe comprises a constant hybridization region complementary to a constant target sequence in the first target nucleic acid. In any of the embodiments herein, the partner probe can comprise an overhang upon hybridization of its constant hybridization region to the constant target sequence in the first target nucleic acid. In any of the embodiments herein, the interrogatory probe can comprise an interrogatory hybridization region complementary to a variant among a plurality of different variants (e.g., wildtype or mutant, major variant or minor variant) of a variable target sequence in the first target nucleic acid. In any of the embodiments herein, the interrogatory probe can comprise a barcode region corresponding to the variant (e.g., a variant-specific barcode region). In some embodiments, the barcode region comprises one or more barcode sequences, and the barcode region or any barcode sequence therein can be a contiguous nucleic acid sequence or a non-contiguous nucleic acid sequence. In any of the embodiments herein, the partner probe can but does not need to comprise a barcode region.

In some of any of the embodiments herein, the biological sample is contacted with a circularizable probe for a second target nucleic acid. In some of any of the embodiments herein, the circularizable probe comprises a hybridization region complementary to a target sequence in the second target nucleic acid. In any of the embodiments herein, the circularizable probe comprises a barcode region corresponding to the second target nucleic acid. In some of any of the embodiments herein, the circularizable probe is a single molecule, such as a single nucleic acid molecule having a contiguous nucleic acid sequence. In some embodiments, the circularizable probe is not provided as multiple molecules which are contacted with the sample and then connected to form a single molecule.

In some of any of the embodiments herein, the method comprises detecting signals associated with the barcode regions or complements thereof (e.g., signals associated with an enzymatic amplification product, such as a rolling circle amplification product (RCP), or signals associated with a non-enzymatic amplification product, such as a branched hybridization complex) at locations in the biological sample, thereby detecting the variant of the first target nucleic acid and detecting the second target nucleic acid at the locations in the biological sample.

In some of any of the embodiments herein, the method comprises circularizing (e.g., comprising ligating) the partner probe and the interrogatory probe hybridized to the first target nucleic acid, thereby generating a first circularized probe comprising the barcode region corresponding to the variant of the first target nucleic acid. In some of any of the embodiments herein, the method comprises circularizing (e.g., comprising ligating) the circularizable probe hybridized to the second target nucleic acid, thereby generating a second circularized probe comprising the barcode region corresponding to the second target nucleic acid. In some of any of the embodiments herein, the method comprises generating an RCP of each of the first and second circularized probes. In some of any of the embodiments herein, the method comprises detecting signals associated with the complements of the barcode regions in the RCPs at locations in the biological sample, thereby detecting the variant of the first target nucleic acid and detecting the second target nucleic acid at the locations in the biological sample.

In some of any of the embodiments herein, the interrogatory hybridization region comprises one or more internal interrogatory nucleotides. For instance, in some embodiments, the interrogatory hybridization region contains a single interrogatory nucleotide that is connected (e.g., via a phosphodiester bond), on both the 3′ and the 5′ of the interrogatory nucleotide, to another nucleotide residue. In some examples, the interrogatory hybridization region contains two or more internal interrogatory nucleotides, and any two or more of the internal interrogatory nucleotides can be contiguous or non-contiguous. In some of any of the embodiments herein, each internal interrogatory nucleotide is complementary to a corresponding nucleotide of interest in the variant. In some embodiments, the interrogatory probe does not comprise a terminal interrogatory nucleotide that has a free 3′ or 5′ terminus and is complementary to a corresponding nucleotide of interest in the variant.

In some of any of the embodiments herein, when the nucleotide at a free 3′ or 5′ terminus of the interrogatory hybridization region is designated as position 1, each of the internal interrogatory nucleotide(s) independently is at nucleotide position 2, 3, 4, 5, 6, 7, 8, 9, 10, 11, 12, 13, 14, 15, 16, 17, 18, 19, 20, or more in the interrogatory hybridization region. In any of the embodiments herein, when the nucleotide at the free 5′ terminus of the interrogatory hybridization region is designated as position 1, each of the internal interrogatory nucleotide(s) can independently be at a nucleotide position between position 5 and position 11, inclusive, in the interrogatory hybridization region. In some embodiments, when the nucleotide at the free 5′ terminus of the interrogatory hybridization region is designated as position 1, each of the internal interrogatory nucleotide(s) can independently be at a nucleotide position between position 3 and position 10, inclusive, in the interrogatory hybridization region.

In any of the embodiments herein, the constant hybridization region may but does not need to comprise an interrogatory nucleotide that is complementary to a corresponding nucleotide of interest in the variant.

In some of any of the embodiments herein, the variant comprises a nucleotide variation, a nucleotide polymorphism, a mutation, a substitution, an insertion, a deletion, a translocation, a duplication, an inversion, and/or a repetitive sequence. In some of any of the embodiments herein, the interrogatory hybridization region comprises a single internal interrogatory nucleotide complementary to a corresponding single nucleotide of interest in the variant, and the single nucleotide of interest can be at the position of a single nucleotide variation (SNV), a single nucleotide polymorphism (SNP), a point mutation, a single nucleotide substitution, a single nucleotide insertion, or a single nucleotide deletion.

In some of any of the embodiments herein, the constant hybridization region in the partner probe or the interrogatory hybridization region in the interrogatory probe is independently of between about 5 and about 50 nucleotides in length. In some of any of the embodiments herein, the constant hybridization region in the partner probe or the interrogatory hybridization region in the interrogatory probe is independently of between about 15 and about 25 nucleotides in length. In some of any of the embodiments herein, the constant hybridization region in the partner probe or the interrogatory hybridization region in the interrogatory probe is independently of about 20 nucleotides in length.

In some of any of the embodiments herein, the constant hybridization region in the partner probe and the interrogatory hybridization region in the interrogatory probe are equal in length. In some of any of the embodiments herein, the constant target sequence and the variable target sequence is equal in length. Alternatively, in some embodiments, the constant hybridization region in the partner probe is shorter or longer than the interrogatory hybridization region in the interrogatory probe, and/or the constant target sequence is shorter or longer than the variable target sequence.

In some of any of the embodiments herein, the partner probe and the interrogatory probe is equal in length. Alternatively, in some embodiments, the partner probe is shorter or longer than the interrogatory probe.

In some of any of the embodiments herein, the overhang in the partner probe comprises a barcode region. In some of any of the embodiments herein, the overhang in the partner probe comprises a barcode region corresponding to the first target nucleic acid. In some of any of the embodiments herein, the partner probe and the interrogatory probe are equal in length and each comprise a barcode region, and the partner probe and the interrogatory probe are symmetric (e.g., the hybridization regions can be equal in length, the barcode regions can be equal in length, and the splint hybridization regions can be equal in length). In some of any of the embodiments herein, the barcode regions in the partner probe and in the interrogatory probe comprises one or more different barcode sequences. In some of any of the embodiments herein, the first circularized probe comprises a barcode sequence from the partner probe and a barcode sequence from the interrogatory probe, and the barcode sequences can be detected independently of each other.

In some of any of the embodiments herein, the barcode region in the interrogatory probe comprises a barcode sequence specific to the variant. In some of any of the embodiments herein, the variant-specific barcode sequence is used to not only distinguish the first target nucleic acid from another nucleic acid such as the second target nucleic acid, but also distinguish a variant (e.g., mutant) from another variant (e.g., wildtype) of the first target nucleic acid. In some of any of the embodiments herein, the partner probe comprises a barcode sequence specific to the first target nucleic acid but not specific to any one or more variants of the variable target sequence in the first target nucleic acid. In some embodiments, the target nucleic acid-specific barcode sequence in the partner probe is configured to distinguish the first target nucleic acid from another nucleic acid such as the second target nucleic acid, but is not configured to distinguish a variant (e.g., mutant) from another variant (e.g., wildtype) of the first target nucleic acid.

In any of the embodiments herein, the overhang in the partner probe may but does not need to comprise a barcode region corresponding to the first target nucleic acid or a sequence thereof. In some embodiments, the overhang in the partner probe does not comprise any barcode sequence. For example, the overhang in the partner probe can consist of one or more common sequences shared by two or more probes targeting different target nucleic acids, such that detection of the overhang or a sequence thereof does not distinguish target nucleic acids having different sequences or distinguish different sequence variants of a target nucleic acid. In some of any of the embodiments herein, the overhang in the partner probe comprises a spacer region that is common among partner probes for two or more target nucleic acids having different sequences.

In some of any of the embodiments herein, the barcode region in the interrogatory probe, the barcode region in the partner probe, and/or the spacer region is independently of between about 4 and about 24 nucleotides in length. In some of any of the embodiments herein, each barcode region is independently of about 16 nucleotides in length, and the spacer region can be about 5 nucleotides in length.

In some of any of the embodiments herein, the partner probe and the interrogatory probe each comprises a splint hybridization region complementary to a splint oligonucleotide, and upon hybridization to the splint oligonucleotide, the splint hybridization regions are configured to be connected, e.g., ligated using the splint oligonucleotide as a template, with or without gap filling and/or cleavage of a 5′ flap prior to the ligation. In some of any of the embodiments herein, the rolling circle amplification product (RCP) of the first circularized probe is performed using the splint oligonucleotide or a portion thereof as a primer, using the first target nucleic acid or a portion thereof as primer, and/or using a primer that is separate from the splint oligonucleotide and the first target nucleic acid.

In some of any of the embodiments herein, the splint hybridization region in the partner probe and the splint hybridization region in the interrogatory probe are independently of between about 2 and about 24 nucleotides in length. In some of any of the embodiments herein, the splint oligonucleotide is between about 8 and about 30 nucleotides in length. In some of any of the embodiments herein, the splint oligonucleotide is between about 20 and about 25 nucleotides in length.

In some of any of the embodiments herein, the splint hybridization region in the partner probe and the splint hybridization region in the interrogatory probe are equal in length. Alternatively, the splint hybridization region in the partner probe can be longer or shorter than the splint hybridization region in the interrogatory probe. In some of any of the embodiments herein, the splint hybridization region in the partner probe and the splint hybridization region in the interrogatory probe each are between about 5 and about 7 nucleotides in length. In some of any of the embodiments herein, the splint oligonucleotide is between about 10 and about 14 nucleotides in length. In some embodiments, the splint oligonucleotide is 12 nucleotides in length, and the splint hybridization region in the partner probe and the splint hybridization region in the interrogatory probe is each 6 nucleotides in length. In some of any of the embodiments herein, the splint hybridization region in the partner probe and the splint hybridization region in the interrogatory probe each are between about 9 and about 11 nucleotides in length. In some of any of the embodiments herein, the splint oligonucleotide is between about 18 and about 22 nucleotides in length. In some embodiments, the splint oligonucleotide is 20 nucleotides in length, and the splint hybridization region in the partner probe and the splint hybridization region in the interrogatory probe is each 10 nucleotides in length.

In some of any of the embodiments herein, the splint hybridization region in the partner probe comprises a barcode region corresponding to the first target nucleic acid or a sequence thereof. In some of any of the embodiments herein, the splint hybridization region in the interrogatory probe comprises a barcode region corresponding to the first target nucleic acid or a sequence thereof. In some of any of the embodiments herein, the splint hybridization region in the partner probe and the splint hybridization region in the interrogatory probe each comprises a portion of a barcode region, where the portions are connected to form the barcode region in the first circularized probe and the barcode region corresponds to the first target nucleic acid or a sequence thereof. In some of any of the embodiments herein, the splint oligonucleotide comprises a complementary barcode region corresponding to the first target nucleic acid or a sequence thereof. In some of any of the embodiments herein, the barcode region(s) in the splint hybridization region in the interrogatory probe and/or the splint hybridization region in the partner probe are specific to the first target nucleic acid. In some of any of the embodiments herein, the barcode region(s) in the splint hybridization region in the interrogatory probe and/or the splint hybridization region in the partner probe are specific to one or more variants of the variable target sequence in the first target nucleic acid, but in some embodiments, this is not required. In some of any of the embodiments herein, the splint hybridization region in the interrogatory probe comprises one or more barcode sequences and/or the splint hybridization region in the partner probe can comprise one or more barcode sequences.

In some of any of the embodiments herein, the splint hybridization region in the interrogatory probe comprises the barcode region corresponding to the variant or a portion of the barcode region. In some of any of the embodiments herein, the splint hybridization region in the interrogatory probe and/or the splint hybridization region in the partner probe comprises a barcode region that does not overlap with or share a common sequence with the barcode region corresponding to the variant. In some of any of the embodiments herein, the barcode region(s) in the splint hybridization region(s) corresponds to the first target nucleic acid and comprise one or more barcode sequences different from barcode sequence(s) in the barcode region corresponding to the variant.

In some embodiments, the splint hybridization region in the interrogatory probe does not comprise a barcode region or a portion thereof, where the barcode region corresponds to the first target nucleic acid or a sequence thereof. In some embodiments, the splint hybridization region in the partner probe does not comprise a barcode region or a portion thereof, where the barcode region corresponds to the first target nucleic acid or a sequence thereof. In some of any of the embodiments herein, the splint hybridization region in the interrogatory probe are common among interrogatory probes for two or more different variants of the first target nucleic acid (e.g., a wildtype sequence and a mutant sequence). In some of any of the embodiments herein, the splint hybridization region in the interrogatory probe is common among interrogatory probes for two or more different target nucleic acids (e.g., the first and second target nucleic acids). In some of any of the embodiments herein, the splint hybridization region in the partner probe is common among partner probes for two or more different target nucleic acids (e.g., the first and second target nucleic acids).

In some of any of the embodiments herein, a pair of interrogatory probe and partner probe are provided for each different variant of the first target nucleic acid, and the splint hybridization regions in the probe pairs for two or more different variants of the first target nucleic acid can be complementary to a common splint oligonucleotide. In some of any of the embodiments herein, a pair of interrogatory probe and partner probe is provided for each different variant of each different target nucleic acid. In some of any of the embodiments herein, the splint hybridization regions in the probe pairs for two or more different variants of the same target nucleic acid are complementary to a common splint oligonucleotide. In some of any of the embodiments herein, the splint hybridization regions in the probe pairs for two or more different target nucleic acids (e.g., including probe pairs for the different variants of each different target nucleic acid) are complementary to a common splint oligonucleotide.

In some of any of the embodiments herein, the circularizable probe for the second target nucleic acid comprises a single hybridization region complementary to the target sequence in the second target nucleic acid. In some embodiments, the single hybridization region has a contiguous nucleic acid sequence and is not a split hybridization region. In some of any of the embodiments herein, the circularizable probe for the second target nucleic acid comprises a 5′ splint hybridization region and a 3′ splint hybridization region. In some embodiments, the 5′ and 3′ splint hybridization regions are complementary to a splint oligonucleotide, and upon hybridization to the splint oligonucleotide, the 5′ and 3′ splint hybridization regions are configured to be ligated using the splint oligonucleotide as a template, with or without gap filling and/or cleavage of a 5′ flap prior to the ligation. In some of any of the embodiments herein, the 5′ splint hybridization region and the 3′ splint hybridization region are equal in length, or the 5′ splint hybridization region can be shorter or longer than the 3′ splint hybridization region. In any of the embodiments herein, the splint oligonucleotide may but does not need to hybridize to the second target nucleic acid. In some embodiments, the splint oligonucleotide hybridizes to a sequence adjacent to the target sequence for the circularizable probe in the second target nucleic acid. In some of any of the embodiments herein, the rolling circle amplification product (RCP) of the second circularized probe is performed using the splint oligonucleotide (that hybridizes to the circularizable probe) or a portion thereof as a primer, using the second target nucleic acid or a portion thereof as primer, and/or using a primer that is separate from the splint oligonucleotide and the second target nucleic acid.

In some of any of the embodiments herein, the circularizable probe for the second target nucleic acid comprised a split hybridization region. In some of any of the embodiments herein, the circularizable probe comprises, from 5′ to 3′: a 5′ target hybridization region, the barcode region corresponding to the second target nucleic acid, and a 3′ target hybridization region. In some of any of the embodiments herein, upon hybridization to the second target nucleic acid, the 5′ and 3′ target hybridization regions are configured to be ligated using the second target nucleic acid as a template, with or without gap filling and/or cleavage of a 5′ flap prior to the ligation. In some of any of the embodiments herein, the 5′ target hybridization region and the 3′ target hybridization region are equal in length. Alternatively, the 5′ target hybridization region can be shorter or longer than the 3′ target hybridization region.

In some of any of the embodiments herein, the barcode region in the circularizable probe comprises a barcode sequence specific to the second target nucleic acid but not specific to any one or more variants of the second target nucleic acid. In some of any of the embodiments herein, the circularizable probe comprises two or more different barcode sequences each corresponding to the second target nucleic acid. In some embodiments, two or more different barcode sequences can be detected independent of each other, e.g., using sequential probe hybridization to each barcode sequence or complement thereof. In some of any of the embodiments herein, the circularizable probe comprises an anchor region that is common among circularizable probes for two or more different target nucleic acids. In some embodiments, a common anchor region is used among a plurality of circularizable probes for two or more different target nucleic acids and a circularized probe generated from ligating a partner probe and an interrogatory probe. In some embodiments, a common anchor region is detected in situ in the biological sample.

In some of any of the embodiments herein, the interrogatory probe for the first target nucleic acid comprises one or more ribonucleotide residues at and/or near a 3′ end. In some of any of the embodiments herein, the partner probe for the first target nucleic acid comprises one or more ribonucleotide residues at and/or near a 3′ end. In some of any of the embodiments herein, the circularizable probe for the second target nucleic acid comprises one or more ribonucleotide residues at and/or near a 3′ end. In some of any of the embodiments herein, the interrogatory probe for the first target nucleic acid comprises a ligatable 3′ ribonucleotide residue. In some of any of the embodiments herein, the partner probe for the first target nucleic acid comprises a ligatable 3′ ribonucleotide residue. In some of any of the embodiments herein, the circularizable probe for the second target nucleic acid comprises a ligatable 3′ ribonucleotide residue. In some of any of the embodiments herein, the interrogatory probe for the first target nucleic acid is composed primarily of DNA and comprise no more than four consecutive ribonucleotide residues. In some of any of the embodiments herein, the partner probe for the first target nucleic acid is composed primarily of DNA and comprise no more than four consecutive ribonucleotide residues. In some of any of the embodiments herein, the circularizable probe for the second target nucleic acid are composed primarily of DNA and comprise no more than four consecutive ribonucleotide residues.

In some of any of the embodiments herein, the partner probe and the interrogatory probe are ligated using the first target nucleic acid as a template, with or without gap filling and/or cleavage of a 5′ flap prior to the ligation. In some of any of the embodiments herein, the first target nucleic acid is an RNA or a DNA, such as an mRNA, cDNA, or genomic DNA. In some of any of the embodiments herein, the second target nucleic acid is an RNA or a DNA, such as an mRNA, cDNA, or genomic DNA. In some of any of the embodiments herein, the first and second target nucleic acids are RNA transcripts of different genes.

In some of any of the embodiments herein, to form the first circularized probe, the ligation of the partner probe and the interrogatory probe templated on the first target nucleic acid and the ligation of the two probes templated on the splint oligonucleotide are performed using the same ligase. In some of any of the embodiments herein, the ligation of the partner probe and the interrogatory probe (either one or both of the first target nucleic acid-templated ligation and the splint oligonucleotide-templated ligation) and the ligation of the circularizable probe templated on the second target nucleic acid (to form the second circularized probe) are performed using the same ligase. In any of the embodiments herein, the ligase can have an RNA-templated ligase activity and/or a DNA-templated ligase activity. In some embodiments, both the first and second target nucleic acids are RNA and a ligase can be used to ligate adjacent, single-stranded DNA (e.g., in probe molecules) splinted by a complementary RNA strand (e.g., the target RNA).

In some of any of the embodiments herein, the ligation of the partner probe and the interrogatory probe and/or the ligation of the circularizable probe is performed using two or more different ligases in the same ligation step or in different ligation steps. The different ligation steps can be performed consecutively, e.g., each using an RNA-templated ligase or a DNA-templated ligase. In some of any of the embodiments herein, the ligation templated on a target RNA (e.g., the first target nucleic acid and/or the second target nucleic acid) is performed using a first ligase having an RNA-templated ligase activity. In some of any of the embodiments herein, the ligation templated on a splint oligonucleotide (e.g., a DNA splint) is performed using a second ligase having a DNA-templated ligase activity. The first and second ligases can be contacted with the biological sample consecutively or simultaneously. For instance, the first and second ligases can be pre-mixed prior to contacting the sample, or added to the sample at the same time in separate compositions. The RNA-templated ligation can be performed before, simultaneously with, or after the DNA-templated ligation.

In some of any of the embodiments herein, the biological sample is contacted with a plurality of different interrogatory probes. In any of the embodiments herein, the plurality of different interrogatory probes can comprise interrogatory probes for the first target nucleic acid and interrogatory probes for one or more other target nucleic acids. In any of the embodiments herein, each different interrogatory probe for the first target nucleic acid can comprise an interrogatory hybridization region complementary to a different variant among the plurality of different variants of the variable target sequence in the first target nucleic acid. In any of the embodiments herein, each different interrogatory probe for the first target nucleic acid can comprise a barcode region corresponding to the different variant.

In some of any of the embodiments herein, the plurality of different variants comprise nucleotides of interest at one or more nucleotide positions in the variable target sequence. In some of any of the embodiments herein, the plurality of different interrogatory probes comprises at least or about 2, at least or about 5, at least or about 10, at least or about 15, at least or about 20, at least or about 25, at least or about 30, at least or about 35, at least or about 40, at least or about 45, at least or about 50, or more different interrogatory probes.

In any of the embodiments herein, a molecule of the first target nucleic acid can comprise a particular variant of the variable target sequence, and two or more different interrogatory probes can compete for hybridization to the particular variant in the molecule. In any of the embodiments herein, a method disclosed herein can comprise washing the biological sample after contacting with the plurality of different interrogatory probes. In some embodiments, the washing comprises a stringent wash. In some embodiments, the washing comprises a less than stringent wash.

In some of any of the embodiments herein, the partner probe, the interrogatory probe, and the circularizable probe each hybridize to its target nucleic acid in situ in the biological sample. In some of any of the embodiments herein, the ligation of the partner probe and the interrogatory probe and the ligation of the circularizable probe are performed in situ in the biological sample. In some of any of the embodiments herein, the rolling circle amplification are performed in situ in the biological sample. In some of any of the embodiments herein, the signals are detected in situ in the biological sample using fluorescent microscopy.

In some of any of the embodiments herein, a method disclosed herein comprises contacting the biological sample with a plurality of nucleic acid probes. In any of the embodiments herein, each nucleic acid probe can comprise a hybridization region complementary to a sequence in one of the RCPs. In any of the embodiments herein, each nucleic acid probe can be detectably labeled or can comprise a detectable region that directly or indirectly binds to a detection oligonucleotide comprising a detectable label. For instance, the detection can comprise using detectably labeled oligonucleotides that directly hybridize to RCPs, and/or detectably labeled oligonucleotides that hybridize to intermediate probes that in turn hybridize to RCPs.

In some of any of the embodiments herein, the plurality of nucleic acid probes comprises a first intermediate probe comprising i) a first hybridization region which hybridizes to the RCP of the first circularized probe for the first target nucleic acid, and ii) a first detectable region; a first detection oligonucleotide that hybridizes to the first detectable region in the first intermediate probe; a second intermediate probe comprising i) a hybridization region which hybridizes to the RCP of the second circularized probe for the second target nucleic acid, and ii) a second detectable region, and a second detection oligonucleotide that hybridizes to the second detectable region in the second intermediate probe.

In some of any of the embodiments herein, the first hybridization region comprises a sequence in the barcode region corresponding to the variant of the first target nucleic acid, and the second hybridization region can comprise a sequence in the barcode region corresponding to the second target nucleic acid. In any of the embodiments herein, the first detectable region and the second detectable region can be the same or different in sequence. In any of the embodiments herein, the first detection oligonucleotide and the second detection oligonucleotide can be the same or different in sequence. In any of the embodiments herein, the first detection oligonucleotide and the second detection oligonucleotide can comprise the same detectable label or different detectable labels. In any of the embodiments herein, in each detection oligonucleotide, the detectable label can correspond to a nucleic acid sequence of the detection oligonucleotide.

In some of any of the embodiments herein, a signal code sequence is assigned to each of i) one or more of the plurality of different variants of the first target nucleic acid and ii) the second target nucleic acid. In any of the embodiments herein, a method disclosed herein can comprise contacting the biological sample with a Cycle 1 intermediate probe and a Cycle 1 detection oligonucleotide to generate a Cycle 1 complex comprising the Cycle 1 intermediate probe hybridized to one of the RCPs and the Cycle 1 detection oligonucleotide hybridized to the Cycle 1 intermediate probe, wherein the Cycle 1 intermediate probe comprises: i) a Cycle 1 hybridization region which hybridizes to the RCP at a sequence complementary to the barcode region corresponding to the variant(s) of the first target nucleic acid or corresponding to the second target nucleic acid, and ii) a Cycle 1 detectable region, and wherein the Cycle 1 detection oligonucleotide comprises: a sequence complementary to the Cycle 1 detectable region, and a Cycle 1 detectable label. In any of the embodiments herein, a method disclosed herein can comprise imaging the biological sample to detect a Cycle 1 signal from the Cycle 1 detectable label, wherein the Cycle 1 signal corresponds to a Cycle 1 signal code in the signal code sequence. In any of the embodiments herein, a method disclosed herein can comprise contacting the biological sample with a Cycle 2 intermediate probe and a Cycle 2 detection oligonucleotide to generate a Cycle 2 complex comprising the Cycle 2 intermediate probe hybridized to the RCP and the Cycle 2 detection oligonucleotide hybridized to the Cycle 2 intermediate probe, wherein the Cycle 2 intermediate probe comprises: i) a Cycle 2 hybridization region which hybridizes to the RCP at the sequence complementary to the barcode region corresponding to the variant(s) of the first target nucleic acid or corresponding to the second target nucleic acid, and ii) a Cycle 2 detectable region, and wherein the Cycle 2 detection oligonucleotide comprises: a sequence complementary to the Cycle 2 detectable region, and a Cycle 2 detectable label. In any of the embodiments herein, a method disclosed herein can comprise imaging the biological sample to detect a Cycle 2 signal from the Cycle 2 detectable label, wherein the Cycle 2 signal corresponds to a Cycle 2 signal code in the signal code sequence, wherein the signal code sequence comprising at least the Cycle 1 signal code and the Cycle 2 signal code is determined based on signals detected at a location in the biological sample, thereby identifying i) the one or more variants of the first target nucleic acid or ii) the second target nucleic acid at the location in the biological sample.

In any of the embodiments herein, the biological sample can be contacted with a pool of Cycle 1 intermediate probes and a universal pool of detection oligonucleotides, wherein each different Cycle 1 intermediate probe comprises i) a hybridization region which hybridizes to an RCP corresponding to a different target nucleic acid or a variant thereof, and ii) a detectable region complementary to a detection oligonucleotide of the universal pool of detection oligonucleotides, wherein the biological sample can be contacted with a pool of Cycle 2 intermediate probes and the universal pool of detection oligonucleotides, wherein each different Cycle 2 intermediate probe comprises i) a hybridization region which hybridizes to an RCP corresponding to a different target nucleic acid or a variant thereof, and ii) a detectable region complementary to a detection oligonucleotide of the universal pool of detection oligonucleotides.

In some of any of the embodiments herein, a method disclosed herein comprises identifying multiple different subsets of the plurality of different variants of the first target nucleic acid in the biological sample, wherein each subset is assigned a different signal code sequence. In some of any of the embodiments herein, each different subset independently contains one or more variants of different sequences. In any of the embodiments herein, a first subset of the variants of the first target nucleic acid can contain a wildtype sequence and can be assigned a first signal code sequence; a second subset of the variants of the first target nucleic acid can contain one, two, three, four, five, or more different mutant sequences and can be assigned a second signal code sequence; and the second target nucleic acid can be assigned a third signal code sequence.

In some of any of the embodiments herein, a method disclosed herein comprises identifying multiple different sequences of multiple different target nucleic acids in the biological sample, wherein each subset of the different sequences of each different target nucleic acid is assigned a different signal code sequence, and each different subset independently contains one or more different sequences.

In any of the embodiments herein, the number of different detection oligonucleotides in the universal pool can be two, three, four, or five. In any of the embodiments herein, each different detection oligonucleotide can comprise a different detectable label corresponding to a nucleic acid sequence of the detection oligonucleotide. In any of the embodiments herein, each different detection oligonucleotide can comprise a different detectable label corresponding a different color in fluorescent microscopy.

In any of the embodiments herein, the pool of Cycle 1 intermediate probes, the pool of Cycle 2 intermediate probes, and the pool(s) of intermediate probes for one or more other cycles can be contacted with the biological sample in sequential cycles in a pre-determined order. The pre-determined order can correspond to the order of the signal codes in the signal code sequences assigned to the barcodes associated with (e.g., via hybridization of barcoded probes to target nucleic acids) the target nucleic acids or sequence variants thereof. In any of the embodiments herein, the sequential cycles can comprise 3, 4, 5, 6, 7, 8, 9, 10, or more cycles.

In some embodiments, disclosed herein is a method for analyzing a biological sample, comprising contacting the biological sample with a partner probe and a plurality of interrogatory probes, wherein the partner probe comprises i) a constant hybridization region complementary to a constant target sequence in a target nucleic acid, and ii) an overhang, wherein each interrogatory probe comprises i) an interrogatory hybridization region complementary to a variant among a plurality of different variants of a variable target sequence in the target nucleic acid, wherein the interrogatory hybridization region comprises one or more internal interrogatory nucleotides, and ii) a barcode region corresponding to the variant, and wherein for a molecule of the target nucleic acid, two or more different interrogatory probes of the plurality of interrogatory probes compete for hybridization to a particular variant of the variable target sequence in the molecule. In some embodiments, the variable target sequence is not at the end of the interrogatory probe. In some embodiments, the plurality of different variants each comprises one or more variant nucleotides that are internal in the variable target sequence. In some embodiments, the method comprises ligating the partner probe and the interrogatory probe hybridized to the molecule of the target nucleic acid, thereby generating a circularized probe comprising a barcode region corresponding to the particular variant. In some embodiments, the method comprises detecting a signal associated with the barcode region in the circularized probe or a complement of the barcode region in an amplification product of the circularized probe, wherein the signal is detected at a location in the biological sample, thereby detecting the particular variant at the location in the biological sample.

In some of any of the embodiments herein, the plurality of interrogatory probes comprises: a first interrogatory probe comprising i) an interrogatory hybridization region comprising one or more internal interrogatory nucleotides complementary to a first sequence of one or more nucleotides in the variable target sequence of the target nucleic acid, and ii) a barcode region corresponding to the first sequence, and a second interrogatory probe comprising i) an interrogatory hybridization region comprising one or more internal interrogatory nucleotides complementary to a second sequence of one or more nucleotides in the variable target sequence of the target nucleic acid, and ii) a barcode region corresponding to the second sequence.

In some of any of the embodiments herein, the first sequence and the second sequence are sequences at the same nucleotide position(s) in the variable target sequence. In any of the embodiments herein, the first sequence can be a wildtype sequence and the second sequence can be a mutant sequence, or vice versa. Alternatively, in any of the embodiments herein, the first sequence can be a major variant sequence and the second sequence can be a minor variant sequence, or vice versa.

In any of the embodiments herein, the first sequence and the second sequence can be sequences at different nucleotide positions in the variable target sequence. In some of any of the embodiments herein, the first sequence is a wildtype or mutant sequence at a first nucleotide position in the variable target sequence, and the second sequence is a wildtype or mutant sequence at a second nucleotide position in the variable target sequence, wherein the first and second nucleotide positions are different (e.g., the first and second nucleotide positions can be different positions in a genetic variant hotspot region).

In some of any of the embodiments herein, the first sequence and the second sequence are independently at the position of a single nucleotide of interest in the variable target sequence. In some of any of the embodiments herein, the signal nucleotide of interest is a single nucleotide variation (SNV), a single nucleotide polymorphism (SNP), a point mutation, a single nucleotide substitution, a single nucleotide insertion, or a single nucleotide deletion.

In some of any of the embodiments herein, the constant hybridization region and the interrogatory hybridization region are equal in length. In some of any of the embodiments herein, the constant target sequence and the variable target sequence are equal in length. In some of any of the embodiments herein, the target nucleic acid is an RNA.

In any of the embodiments herein, a different signal code sequence can be assigned to each different variant, and the method can comprise contacting the biological sample with a Cycle 1 intermediate probe and a Cycle 1 detection oligonucleotide to generate a Cycle 1 complex comprising the Cycle 1 intermediate probe hybridized to the barcode region or a complement thereof and the Cycle 1 detection oligonucleotide hybridized to the Cycle 1 intermediate probe, wherein the Cycle 1 intermediate probe comprises: i) a Cycle 1 hybridization region which hybridizes to the barcode region or complement thereof, and ii) a Cycle 1 detectable region, and wherein the Cycle 1 detection oligonucleotide comprises: a sequence complementary to the Cycle 1 detectable region, and a Cycle 1 detectable label. In any of the embodiments herein, the method can comprise imaging the biological sample to detect a Cycle 1 signal from the Cycle 1 detectable label, wherein the Cycle 1 signal corresponds to a Cycle 1 signal code in the signal code sequence. In any of the embodiments herein, the method can comprise contacting the biological sample with a Cycle 2 intermediate probe and a Cycle 2 detection oligonucleotide to generate a Cycle 2 complex comprising the Cycle 2 intermediate probe hybridized to the barcode region or complement thereof and the Cycle 2 detection oligonucleotide hybridized to the Cycle 2 intermediate probe, wherein the Cycle 2 intermediate probe comprises: i) a Cycle 2 hybridization region which hybridizes to the barcode region or complement thereof, and ii) a Cycle 2 detectable region, and wherein the Cycle 2 detection oligonucleotide comprises: a sequence complementary to the Cycle 2 detectable region, and a Cycle 2 detectable label. In any of the embodiments herein, the method can comprise imaging the biological sample to detect a Cycle 2 signal from the Cycle 2 detectable label, wherein the Cycle 2 signal corresponds to a Cycle 2 signal code in the signal code sequence, wherein the signal code sequence comprising at least the Cycle 1 signal code and the Cycle 2 signal code is determined based on signals detected at a location in the biological sample, thereby identifying the particular variant of the target nucleic acid at the location in the biological sample. In any of the embodiments herein, the amplification product of the circularized probe can be a rolling circle amplification product (RCP). In any of the embodiments herein, for each different variant of the target nucleic acid, a corresponding RCP can be generated in the biological sample.

In any of the embodiments herein, the method can comprise contacting the biological sample with a pool of Cycle 1 intermediate probes and a universal pool of detection oligonucleotides, wherein each different Cycle 1 intermediate probe comprises i) a hybridization region which hybridizes to an RCP corresponding to a different variant of the target nucleic acid, and ii) a detectable region complementary to a detection oligonucleotide of the universal pool of detection oligonucleotides, wherein the biological sample can be contacted with a pool of Cycle 2 intermediate probes and the universal pool of detection oligonucleotides, wherein each different Cycle 2 intermediate probe comprises i) a hybridization region which hybridizes to the RCP, and ii) a detectable region complementary to a detection oligonucleotide of the universal pool of detection oligonucleotides.

In some of any of the embodiments herein, a mutant sequence of the variable target sequence corresponds to a barcode region in a corresponding interrogatory probe and is assigned a first signal code sequence. In some of any of the embodiments herein, a wildtype sequence or a different mutant sequence of the variable target sequence corresponds to a barcode region in a corresponding interrogatory probe and is assigned a second signal code sequence different from the first signal code sequence.

In some of any of the embodiments herein, each barcode region independently comprises one, two, three, or more barcode sequences configured to be detected independently of one another. In some of any of the embodiments herein, the barcode region corresponding to the variant comprises two barcode sequences configured to be detected independently of each another. In any of the embodiments herein, each barcode region or a barcode sequence therein can be independently a contiguous nucleic acid sequence or a non-contiguous nucleic acid sequence.

In some of any of the embodiments herein, the biological sample comprises a cell or tissue sample comprising cells or cellular components. In some of any of the embodiments herein, the biological sample is a tissue section. In some of any of the embodiments herein, the biological sample is a formalin-fixed, paraffin-embedded (FFPE) sample, a frozen tissue sample, or a fresh tissue sample. In some of any of the embodiments herein, the biological sample is fixed and/or permeabilized. In some of any of the embodiments herein, the biological sample is crosslinked and/or embedded in a matrix. In some embodiments, the matrix comprises a hydrogel. In any of the embodiments herein, the biological sample can be cleared.

In some embodiments, disclosed herein is a kit for analyzing a biological sample, comprising a partner probe and an interrogatory probe for a first target nucleic acid, wherein the partner probe comprises i) a constant hybridization region complementary to a constant target sequence in the first target nucleic acid, and ii) an overhang, wherein the interrogatory probe comprises i) an interrogatory hybridization region complementary to a variant among a plurality of different variants of a variable target sequence in the first target nucleic acid, and ii) a barcode region corresponding to the variant. In some embodiments, the kit comprises a circularizable probe for a second target nucleic acid, comprising i) a hybridization region complementary to a target sequence in the second target nucleic acid, and ii) a barcode region corresponding to the second target nucleic acid, wherein the circularizable probe is a single molecule.

In some embodiments, disclosed herein is a kit for analyzing a biological sample, comprising a partner probe and a plurality of interrogatory probes, wherein the partner probe comprises i) a constant hybridization region complementary to a constant target sequence in a target nucleic acid, and ii) an overhang; wherein each interrogatory probe comprises i) an interrogatory hybridization region complementary to a variant among a plurality of different variants of a variable target sequence in the target nucleic acid, wherein the interrogatory hybridization region comprises one or more internal interrogatory nucleotides, and ii) a barcode region corresponding to the variant; and wherein two or more different interrogatory probes of the plurality of interrogatory probes are configured to compete for hybridization to a particular variant of the variable target sequence in a molecule of the target nucleic acid.

In some of any of the embodiments herein, the kit comprises one or more reagents for probe circularization, one or more reagents for rolling circle amplification (RCA) of a circularized probe, and/or one or more reagents for detecting an RCA product (RCP). In some of any of the embodiments herein, the kit comprises instructions for using the kit components to perform a method disclosed herein.

### BRIEF DESCRIPTION OF THE DRAWINGS

The drawings illustrate certain embodiments of the features and advantages of this disclosure. These embodiments are not intended to limit the scope of the appended claims in any manner.

FIGS. 1A-1D show RCA-based detection of sequence variants in situ. Using SNP detection as an example, hybridization and circularization of probes can lead to correct readout of the sequence variant (FIG. 1A), incorrect readout (FIG. 1B), or a non-viable ligation product (FIG. 1C). FIG. 1D shows exemplary probes for in situ detection of sequence variants (e.g., SNP 1, SNP 2, or SNP 3) comprising a partner probe and an interrogatory probe.

FIGS. 2A-2C show exemplary probe pairs each comprising an interrogatory probe and a partner probe for analyzing a target nucleic acid.

FIGS. 3A-3B show exemplary probes for analyzing a first target nucleic acid and a second target nucleic acid. The probe pair comprises an interrogatory probe and a partner probe as shown in FIG. 2 for analyzing a variant of the first target nucleic acid, and the circularizable probe for analyzing the second target nucleic acid can comprise a split hybridization region for target hybridization (FIG. 3A) or a single contiguous target hybridization region (FIG. 3B).

FIG. 4A shows probe pairs (Pos5 and Pos11 having the interrogatory nucleotide at position 5 and position 11, respectively, in the interrogatory arm from the ligation site with the partner probe) for in situ detection of KRAS wildtype and mutant alleles. The partner probes may each comprise an optional spacer region and/or an optional barcode region. FIG. 4B shows detected RCP counts per nuclei area in two different cell lines, ME-180 (KRAS wildtype) and A549 (KRAS mutant), using both Pos5 and Pos11.

FIG. 5A shows KRAS sequence variants targeted by probe pairs (“WT” for KRAS wildtype, “MUT” for KRAS mutant c.38G>A, and “Hotspots” for other hotspot mutations) for in situ detection of the KRAS alleles. FIG. 5B shows detected RCP counts per nuclei area in the A549 (KRAS mutant) cell line, using probe pairs comprising an interrogatory probe targeting the WT, MUT, and Hotspots sequence variants. The in situ allele detection using probe pairs was compatible with detection using circularizable probes having symmetric target hybridization arms and maintained specificity of allele detection.

FIG. 6 depicts probe pairs for allele detection (e.g., SNP probes) and a panel of circularizable probes (e.g., hBreast panel probes) for gene transcript detection in situ in FFPE human breast tissue samples. An optional SNP anchor in the partner probe of the probe pairs and an optional anchor in the circularizable probe are shown.

FIG. 7 is an example workflow of analysis of a biological sample (e.g., a cell or tissue sample) using an opto-fluidic instrument, according to various embodiments.

### DETAILED DESCRIPTION

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In some embodiments, provided herein are methods and compositions that reduce false positive signals (e.g., due to incorrect probe hybridization and/or ligation) and/or increase detection efficiency of true positive signals for the identification of variant sequences (e.g., SNVs, SNPs or point mutations) in situ in cell or tissue samples. In some embodiments, provided herein is a method for in situ nucleic acid detection that may utilize discrimination of a variant sequence during target recognition (e.g., via probe hybridization to a nucleic acid analyte) and subsequent probe ligation (e.g., circularization of a circularizable probe using the nucleic acid analyte as template).

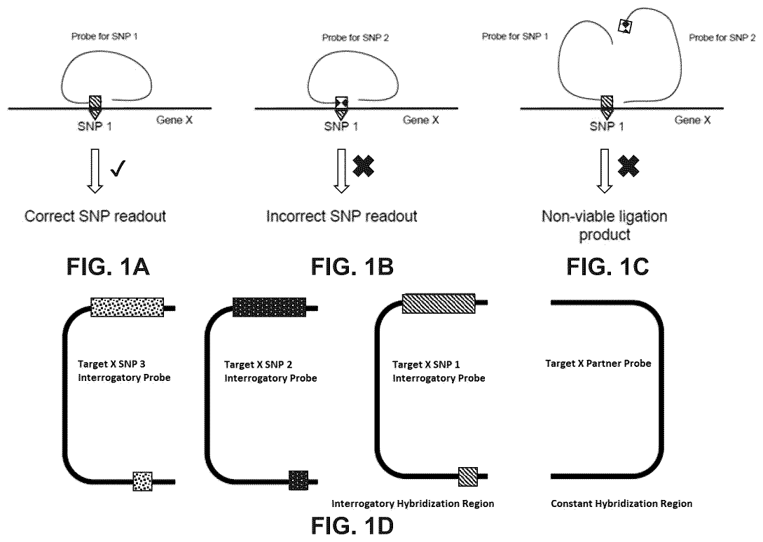

As shown in FIGS. 1A-1C, a circularizable probe having an interrogatory hybridization region (e.g., for hybridizing to and interrogating a variable target sequence for one or more sequence variants, such as an SNP-specific binding arm) physically linked to a constant hybridization region (e.g., a non-SNP binding arm) may not be optimal for detecting sequence variants in situ. For instance, probes having different SNP-specific binding arms may not be able to effectively compete for binding to the SNP sequences when they share the same constant non-SNP binding arm, since the non-SNP binding arm hybridized to a conserved sequence can limit the mobility of the physically linked SNP-specific binding arms (which may different from each other by a single nucleotide), resulting in approximately the same hybridization affinity across probes designed for the same target nucleic acid but for different SNP sequence variants.

In some embodiments, a method disclosed herein comprises hybridizing a partner probe (e.g., a constant probe) to a conserved region (e.g., a constant target sequence) in a target nucleic acid, and an interrogatory probe to a variable target sequence (e.g., a mutation hotspot region) in the target nucleic acid. In some embodiments, the partner probe and the interrogatory probe forms a probe pair wherein the probe pair contains a split target hybridization region as well as a split splint hybridization region. For example, a probe pair comprises an SNP-interrogating probe with an interrogatory nucleotide (e.g., for detecting a mutation) in a target-binding arm and a SNP-specific barcode sequence in a region that does not bind to the target nucleic acid. In some embodiments, the other probe of the probe pair comprises a constant target-binding arm (e.g., having no interrogatory nucleotide such as nucleotides complementary to mutations to be detected). The other probe may but does not need to contain a barcode sequence. Since the SNP-interrogating probe and the partner probe are provided as physically separate molecules for hybridizing to the target nucleic acid, the SNP-interrogating probes for correct SNPs can more effectively and efficiently outcompete probes for incorrect SNPs, thereby increasing the chances of correct probe hybridization and circularization and eventually correct RCA-based readout. Using SNP detection as an example, as shown in FIG. 1D, the target X SNP interrogatory probes can be specific to each SNP, and multiple SNP interrogatory probes each comprising an SNP-specific target binding arm and a corresponding SNP-specific barcode sequence can compete for hybridization to a particular SNP sequence variant, allowing the best matching SNP interrogatory probe to outcompete other SNP interrogatory probes without hindrance by the physically separate partner probe hybridizing to a constant target sequence. In some embodiments, the best matching SNP interrogatory probe bound to the target nucleic acid is ligated to the constant hybridization region (e.g., a stable binding arm) of a partner probe (e.g., a target X constant probe).

In some embodiments, a constant target sequence is present in the majority or all RNA transcripts from the same gene (e.g., KRAS), and the variable target sequence in the RNA transcripts may comprise one or more variant sequences (each of one or more bases), or regions to be interrogated (e.g., one or more SNPs) depending on the particular transcript. In some embodiments, the variable target sequences comprise mutation hotspots.

In some embodiments, the interrogatory hybridization regions of the interrogatory probes compete with one another for hybridization to a variable target sequence that contains particular variant sequence(s). For instance, in the case of KRAS, mutations occur most frequently in 5 bases in codons 12 and 13. In some embodiments, a library of interrogatory probes is designed to the possible variants (or any subset thereof) in that region with a length of about 12 bases. In some embodiments, the interrogatory hybridization regions is between about 6 and about 18 bases. In some embodiments, with short interrogatory hybridization region, a single mismatch of one base, especially when the mismatch is internal within a short interrogatory hybridization region, can punish and reduce the stability of the hybridization and the fully correct matching interrogatory hybridization region to the variant of the target nucleic acid is favored. The length of the variant-interrogating binding arm can be reduced to improve sequence variant discrimination ability of the probe pair. Compared to a one-piece circularizable probe strategy where one arm of the probe matches a conserved region in the target sequence and a mismatch on the other arm (e.g., the arm containing an SNP-interrogatory nucleotide) does not affect the arm matching the conserved region, the probe pair approach comprising two separate probe oligonucleotides allows competition among interrogatory hybridization regions in different interrogatory probes and dissociation of mismatched interrogatory probes prior to probe ligation.

In some embodiments, the ligation of the interrogatory probe and the partner probe is performed using RNA-templated ligation. In some embodiments, the ligation is performed after hybridization of a plurality of interrogatory probes to target nucleic acids in a sample and removing interrogatory probes mismatched on target nucleic acids, and the method comprises ligating an interrogatory probe matched with a target nucleic acid to the partner probe hybridized to the same target nucleic acid. In some embodiments, the ligation is performed simultaneously with the interrogatory probe and the partner probe hybridization, e.g., a ligase is present during probe hybridization. In some embodiments, the interrogatory probe and the partner probe (e.g., as part of a plurality of interrogatory probes and a plurality of partner probes) are contacted with the sample simultaneously or in any order. In some embodiments, the sample is contacted with the plurality of interrogatory probes and the plurality of partner probes at the same time, and the plurality of interrogatory probes and the plurality of partner probes are pre-mixed or not pre-mixed prior to contacting the sample. In some embodiments, the plurality of partner probes are hybridized to target nucleic acids in the sample before the interrogatory probes are hybridized. In some embodiments, the interrogatory probes are hybridized to target nucleic acids in the sample before the plurality of partner probes are hybridized.

In some embodiments, the plurality of interrogatory probes and the plurality of partner probes are for detecting sequence variants of a first target nucleic acid, the plurality of partner probes of the same sequence, and the plurality of interrogatory probes comprise interrogatory probes each targeting a different sequence variant of the first target nucleic. In some embodiments, the biological sample is contacted with a circular or circularizable probe for detecting a second target nucleic acid. In some embodiments, a plurality of interrogatory probes (e.g., a plurality of different interrogatory probes) and a plurality of partner probes for detecting sequence variants of a first target nucleic acid, and circular or circularizable probes for detecting one or more other target nucleic acids (e.g., a second target nucleic acid and/or a third nucleic acid, etc.) are contacted with the sample simultaneously or in any order. Provided herein are assays using a combination of two-oligonucleotide molecule probe pairs (e.g., comprising partner probes and interrogatory probes) and one-oligonucleotide molecule probes (e.g., circular or circularizable probe). Also provided herein in some aspects is a kit comprising a panel of two-oligonucleotide molecule probe pairs (e.g., each different pair targeting different variant sequences of a first target nucleic acid, such as SNVs, SNPs, or mutations) and a panel of one-oligonucleotide molecule probes (e.g., each different one-oligonucleotide molecule probe targeting a different target nucleic acid, such as transcripts of different genes of interest). The panels of probes can be combined or contacted with a biological sample in any order, and the RCPs generated from the probes after probe circularization in the sample can be detected at locations in the sample and the barcodes in the RCPs can be decoded using sequential hybridization of probes to the RCPs, thereby identifying the variant sequences and target nucleic acids at the locations in the sample.

In some embodiments, one or more washes are performed between any of interrogatory probe hybridization, partner probe hybridization, circular or circularizable probe hybridization, and ligation. In some embodiments, any one or more of the washes is/are stringent so that only completely complementary probes remain bound to target nucleic acids after the wash(es). In some embodiments, any one or more of the washes is/are performed under less than stringent conditions. In some embodiments, any one or more of the washes is/are performed under extremely low stringency conditions, low stringency conditions, or medium stringency conditions.

In some embodiments, disclosed herein is a method for analyzing a biological sample, comprising: a) contacting the biological sample with: i) a partner probe and an interrogatory probe for a first target nucleic acid, wherein the partner probe comprises i) a constant hybridization region complementary to a constant target sequence in the first target nucleic acid, and ii) an overhang, wherein the interrogatory probe comprises i) an interrogatory hybridization region complementary to a variant among a plurality of different variants of a variable target sequence in the first target nucleic acid, and ii) a barcode region corresponding to the variant, and ii) a circular or circularizable probe for a second target nucleic acid, comprising i) a hybridization region complementary to a target sequence in the second target nucleic acid, and ii) a barcode region corresponding to the second target nucleic acid; b) ligating the partner probe and the interrogatory probe hybridized to the first target nucleic acid, thereby generating a circularized probe comprising the barcode region corresponding to the variant of the first target nucleic acid; c) generating a rolling circle amplification product (RCP) of each of: the circularized probe comprising the barcode region corresponding to the variant of the first target nucleic acid, and the circular probe or a circularized probe (generated from the circularizable probe) comprising the barcode region corresponding to the second target nucleic acid; and d) detecting signals associated with the complements of the barcode regions in the RCPs at locations in the biological sample, thereby detecting the variant of the first target nucleic acid and detecting the second target nucleic acid at the locations in the biological sample.

In some embodiments, the interrogatory hybridization region comprises a single internal interrogatory nucleotide complementary to a corresponding single nucleotide of interest in the variant, and the single nucleotide of interest is at the position of a single nucleotide variation (SNV), a single nucleotide polymorphism (SNP), a point mutation, a single nucleotide substitution, a single nucleotide insertion, or a single nucleotide deletion. In some embodiments, the RCPs comprising complements of the barcode regions (a barcode region can correspond to one or more variants of the first target nucleic acid, or correspond to the second target nucleic acid) are detected using nucleic acid probes contacted with the biological sample in sequential cycles in a pre-determined order. The pre-determined order can correspond to the order of the signal codes in the signal code sequences assigned to the barcodes associated with (e.g., via hybridization of barcoded probes to target nucleic acids) the target nucleic acids or sequence variants thereof. As such, in situ detection of sequence variants (e.g., alleles of the same gene or transcript) can be compatible with in situ detection of different target nucleic acids (e.g., different genes or transcripts) in the sample biological sample.

### II. Nucleic Acid Analytes and Target Sequences

In some aspects, provided herein are methods and compositions for analysis of target nucleic acids. In some embodiments, the target nucleic acids comprise RNA. In some embodiments, the target nucleic acids comprise genomic DNA. In some embodiments, the target nucleic acids comprise cDNA. In some embodiments, one or more target nucleic acids each comprises a variable target sequence (e.g., variant sequence) of one or more nucleotides. In some embodiments, one or more target nucleic acids each comprises a variant sequence of one or more nucleotides. In some embodiments, one or more target nucleic acids each comprises a single-nucleotide polymorphism (SNP). In some embodiments, one or more target nucleic acids each comprises is a single-nucleotide variant (SNV). In some embodiments, one or more target nucleic acids each comprises a single-nucleotide substitution. In some embodiments, one or more target nucleic acids each comprises a point mutation. In some embodiments, one or more target nucleic acids each comprises a single-nucleotide insertion. In some embodiments, one or more target nucleic acids each comprises a single-nucleotide deletion. In some embodiments, target genomic DNA, target RNA, and/or target cDNA comprising one or more sequence variants at one or more genomic loci are analyzed as described herein. In some embodiments, target genomic DNA, target RNA, and/or target cDNA comprising one or more single-nucleotide differences (e.g., SNPs, SNVs, point mutations, etc.) at one or more genomic loci are analyzed, and the identity of one or more single-nucleotide differences is determined in situ in a sample.

In some embodiments, the target nucleic acid comprises a nucleotide variation, a nucleotide polymorphism, a mutation, a substitution, an insertion, a deletion, a translocation, a duplication, an inversion, and/or a repetitive sequence, in a variant sequence among a plurality of different sequences to be identified in situ in a biological sample. In some embodiments, the variant sequence is a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the variant sequence comprises multiple nucleotides, and each nucleotide is independently at the position of an SNV, an SNP, a point mutation, a single nucleotide substitution, a single nucleotide insertion, or a single nucleotide deletion. In some embodiments, the target nucleic acid is an RNA, such as an miRNA or a transcript of an oncogene, a tumor suppressor gene, an immune gene, or an antigen receptor gene.

The methods, probes, and kits disclosed herein can be used to detect and analyze a wide variety of different analytes. Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to allow access of one or more reagents (e.g., probes for analyte detection) to the analytes in the cell or cell compartment or organelle.

Analytes of particular interest may include nucleic acid molecules (e.g., cellular nucleic acids), such as DNA (e.g. genomic DNA, cDNA, mitochondrial DNA, plastid DNA, viral DNA, etc.) and RNA (e.g. mRNA, microRNA, rRNA, snRNA, viral RNA, etc.), and synthetic and/or modified nucleic acid molecules (e.g. including nucleic acid domains comprising or consisting of synthetic or modified nucleotides such as LNA, PNA, morpholino, etc.).

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods, probes, and kits disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises or is associated with a target sequence. In some embodiments, the target nucleic acid and the target sequence therein is endogenous to the sample, generated in the sample, added to the sample, or associated with an analyte in the sample. In some embodiments, the target sequence is a single-stranded target sequence (e.g., a sequence in a rolling circle amplification product). In some embodiments, the target sequence is a single-stranded target sequence (e.g., in a probe bound directly or indirectly to the analyte). In some embodiments, the target sequence is a single-stranded target sequence in a primary probe that binds to an analyte of interest in the biological sample. In some embodiments, the target sequence is a single-stranded target sequence in an intermediate probe which directly or indirectly binds to a primary probe or product thereof, where the primary probe binds to an analyte of interest in the biological sample. In some embodiments, the target sequence is a single-stranded target sequence in a secondary probe that binds to the primary probe or product thereof. In some embodiments, the analytes comprise one or more single-stranded target sequences.

In some embodiments, provided herein are methods for in situ analysis of a target nucleic acid. In some embodiments, the in situ analysis comprises using different probes (e.g., interrogatory probes) to compete for hybridization to the target nucleic acid. In some embodiments, the interrogatory probes are ligated to partner probes to form circularized probes. In some embodiments, the circularized probes are amplified (e.g., using RCA) and the amplicons are detected in situ.

In some embodiments, probes for in situ analysis comprises partner probes (e.g. constant probes) that target common/constant regions adjacent to hotspots for mutation. In some embodiments, the common/constant region is adjacent to a variable target sequence in the target nucleic acid. In some embodiments, the variable target sequence comprises one or more hotspots for mutation. In some embodiments, the variable target sequence comprises a variant sequence among a plurality of different variant sequences. In some embodiments, the variant sequence is a major variant sequence. In some embodiments, the variant sequence is a minor variant sequence. In some embodiments, the variable target sequence is targeted by a library of interrogatory probes, which are incubated with the sample for hybridization to target nucleic acid molecules, allowing the best matching interrogatory probe to outcompete other interrogatory probes in the library. In some embodiments, after washing the sample, the best matching interrogatory probes (e.g., highest complementarity) are ligated to the adjacently hybridized partner probes to form circularized probes which are amplified.

In some embodiments, amplicons (e.g., RCPs) comprising the variants of the variable target sequences, barcode regions corresponding to the variants of the variable target sequence, or complements thereof, are detected in situ using sequencing-by-synthesis, sequencing-by-ligation, sequencing-by-binding, avidity sequencing, or sequential hybridization of probes. In some embodiments, the RCA products are sequentially contacted with pools of intermediate probes, where each intermediate probe comprises (i) a hybridization sequence for recognition of a copy of a variable target sequence, a barcode region, or complement thereof, in an RCP, and (ii) a detectable region, such as an overhang sequence, for hybridization of a detectably labeled oligonucleotide.

### III. Probes and Probe Circularization

In some embodiments, provided herein are nucleic acid probes (e.g., partner probes and interrogatory probes) that can be circularized. In some embodiments, probes are hybridized to a target nucleic acid molecule comprising a variable target sequence which comprises one of multiple variant sequences, and the probes are circularized to generate a circularized probe. In some embodiments, the circularized probe comprising at least portions of the complement of the variable target sequence are amplified (e.g., using RCA) and the amplification product is detected in order to detect the variant sequence in the target nucleic acid molecule.

In some embodiments, a method disclosed herein comprises contacting a biological sample with a plurality of nucleic acid probes. In some embodiments, each nucleic acid probe comprises a hybridization region complementary to a sequence in one of an RCPs. In some embodiments, each nucleic acid probe is detectably labeled or comprises a detectable region that directly or indirectly binds to a detection oligonucleotide comprising a detectable label. For instance, in some cases detecting the RCPs comprises contacting the sample with detectably labeled oligonucleotides that directly hybridize to the RCPs, and detecting the detectably labeled oligonucleotides. In some cases, detecting the RCPs comprises contacting the sample with detectably labeled oligonucleotides that hybridize to intermediate probes that in turn hybridize to RCPs, and detecting the detectably labeled oligonucleotides.

In some embodiments, disclosed herein is a method for analyzing a biological sample, the method comprising contacting the biological sample with a partner probe and an interrogatory probe for a target nucleic acid (e.g., a first target nucleic acid). In some embodiments, the target nucleic acid comprises a constant target sequence and a variable target sequence. While the constant target sequence is common among a plurality of different variants of the target nucleic acid, the sequences of the variable target sequence in the plurality of different variants can differ at one or more nucleotide positions in the variable target sequence. In some embodiments, the partner probe comprises a constant hybridization region complementary to the constant target sequence in the target nucleic acid. In some embodiments, the partner probe comprises a 5′ or 3′ overhang upon hybridization of its constant hybridization region to the constant target sequence in the target nucleic acid. In some embodiments, the 5′ or 3′ overhang in the partner probe comprises a barcode region corresponding to a first target nucleic acid. In some embodiments, the interrogatory probe comprises an interrogatory hybridization region complementary to a variant among a plurality of different variants (e.g., wildtype or mutant, major variant or minor variant) of the variable target sequence in the target nucleic acid. In some embodiments, the interrogatory probe comprises a barcode region comprising one or more barcode sequences corresponding to the variant. In some embodiments, the barcode regions in the partner probe and in the interrogatory probe comprise one or more different barcode sequences. In some embodiments, the barcode region is a variant-specific barcode region. In some embodiments, the barcode region contains one or more barcode sequences. In some embodiments, the barcode region or any barcode sequence therein is a contiguous nucleic acid sequence or a non-contiguous nucleic acid sequence. In some embodiments, the barcode region comprises a plurality of contiguous barcode sequences.

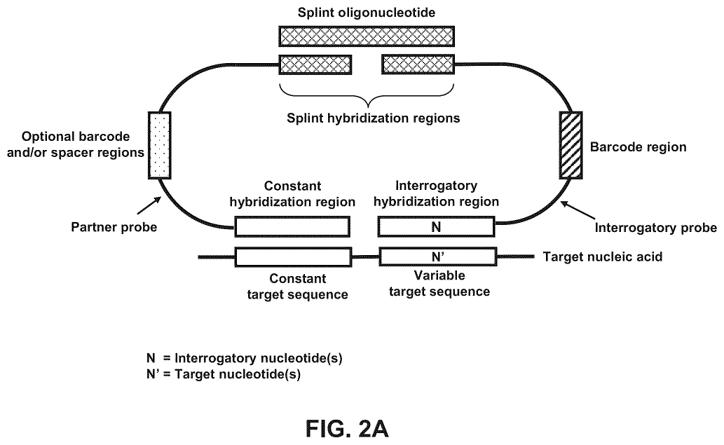

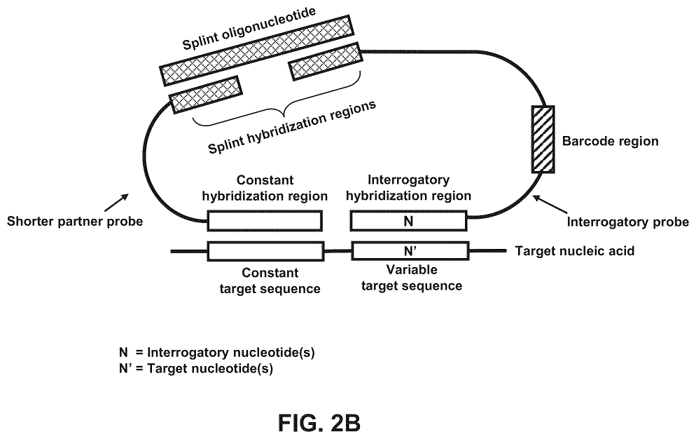

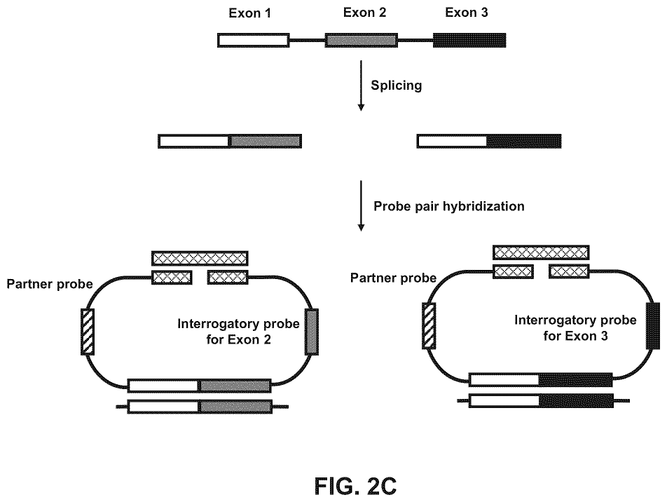

As shown in FIG. 2A, an exemplary probe pair can comprise a partner probe and an interrogatory probe configured to hybridize to a target nucleic acid. In some examples, the target nucleic acid comprises, from 5′ to 3′: a constant target sequence and a variable target sequence which comprises one or more target nucleotides (nucleotides of interest). In some embodiments, the partner probe comprises, from 5′ to 3′: a constant hybridization region complementary to the constant target sequence; one or more optional barcode regions and/or one or more spacer regions, which barcode region(s) and spacer region(s) can be arranged in any order; and a splint hybridization region (e.g., a 3′ splint hybridization region having a ligatable 3′ terminal nucleotide residue). In some embodiments, the interrogatory probe comprises, from 5′ to 3′: a splint hybridization region (e.g., a 5′ splint hybridization region having a ligatable 5′ terminal nucleotide residue); a barcode region corresponding to the variable target sequence comprising the target nucleotide(s); and an interrogatory hybridization region complementary to the variable target sequence. In some examples, the target nucleic acid comprises, from 5′ to 3′: a variable target sequence which comprises one or more target nucleotides (nucleotides of interest); and a constant target sequence. In some embodiments, the partner probe comprises, from 5′ to 3′: a splint hybridization region (e.g., a 5′ splint hybridization region having a ligatable 5′ terminal nucleotide residue); one or more optional barcode regions and/or one or more spacer regions, which barcode region(s) and spacer region(s) can be arranged in any order; and a constant hybridization region complementary to the constant target sequence. In some embodiments, the interrogatory probe comprises, from 5′ to 3′: an interrogatory hybridization region complementary to the variable target sequence; a barcode region corresponding to the variable target sequence comprising the target nucleotide(s); and a splint hybridization region (e.g., a 3′ splint hybridization region having a ligatable 3′ terminal nucleotide residue). The partner probe and the interrogatory probe in FIG. 2A can be equal in length. In some examples, the interrogatory probe and the partner probe each comprises a 5′ phosphate for ligation. In some embodiments, the barcode region in the interrogatory probe comprises one or more barcode sequences, and the barcode region in the partner probe comprises one or more barcode sequences. In some embodiments, the interrogatory probe barcode region (or a portion thereof) is in the splint hybridization region of the interrogatory probe. In some embodiments, the constant probe barcode region (or a portion thereof) is in the splint hybridization region of the constant probe.

FIG. 2B shows an exemplary probe pair comprising a partner probe and an interrogatory probe configured to hybridize to a target nucleic acid, where the partner probe is shorter than the interrogatory probe. In some examples, the target nucleic acid comprises, from 5′ to 3′: a constant target sequence and a variable target sequence which comprises one or more target nucleotides (nucleotides of interest). In some embodiments, the partner probe comprises, from 5′ to 3′: a constant hybridization region complementary to the constant target sequence; an optional spacer region and no barcode region; and a splint hybridization region. In some embodiments, the interrogatory probe comprises, from 5′ to 3′: a splint hybridization region; a barcode region corresponding to the variable target sequence comprising the target nucleotide(s); and an interrogatory hybridization region complementary to the variable target sequence. In some examples, the target nucleic acid comprises, from 5′ to 3′: a variable target sequence which comprises one or more target nucleotides (nucleotides of interest); and a constant target sequence. In some embodiments, the partner probe comprises, from 5′ to 3′: a splint hybridization region; an optional spacer region and no barcode region; and a constant hybridization region complementary to the constant target sequence. In some embodiments, the interrogatory probe comprises, from 5′ to 3′: an interrogatory hybridization region complementary to the variable target sequence; a barcode region corresponding to the variable target sequence comprising the target nucleotide(s); and a splint hybridization region. In some embodiments, the partner probe consists of the constant hybridization region and the splint hybridization region. In some embodiments, the partner probe contains no spacer region or barcode region. In some embodiments, the partner probe contains no spacer region or barcode region between the constant hybridization region and the splint hybridization region. In other words, the constant hybridization region and the splint hybridization region of the partner probe can be directly linked via a phosphodiester bond.

In some embodiments, the variable target sequence is about or at least 4, about or at least 6, about or at least 8, about or at least 10, about or at least 12, about or at least 14, about or at least 16, about or at least 18, about or at least 20, or more nucleotides in length. In some embodiments, the variable target sequence comprises a variant sequence among a plurality of different variant sequences.

In some embodiments, a target nucleotide (e.g., as shown in FIGS. 2A-2B) is at least 1, 2, 3, 4, 5, 6, 7, 8, 9, or more phosphodiester bonds from the 3′ or 5′ end of the variable target sequence. In some embodiments, an interrogatory nucleotide (e.g., as shown in FIGS. 2A-2B) is at least 1, 2, 3, 4, 5, 6, 7, 8, 9, or more phosphodiester bonds from a ligatable 5′ or 3′ end of the interrogatory hybridization region. In some embodiments, a target nucleotide is at least 3 or more phosphodiester bonds from the 3′ or 5′ end of the variable target sequence. In some embodiments, an interrogatory nucleotide is at least 3 or more phosphodiester bonds from a ligatable 5′ or 3′ end of the interrogatory hybridization region. In some embodiments, an interrogatory nucleotide is at least 3 or more phosphodiester bonds from a ligatable 5′ or 3′ end of the interrogatory hybridization region in an interrogatory probe with an interrogatory hybridization region that is 20 nucleotides in length. In some embodiments, an interrogatory nucleotide is at least 5 or more phosphodiester bonds from a ligatable 5′ or 3′ end of the interrogatory hybridization region in an interrogatory probe with an interrogatory hybridization region that is 20 nucleotides in length.

In some embodiments, the target nucleotide is at or near the central nucleotide(s) of the variable target sequence. In some embodiments, the interrogatory nucleotide is at or near the central nucleotide(s) of the interrogatory hybridization region. In some embodiments, the interrogatory nucleotide is no more than 1, no more than 2, no more than 3, no more than 4, no more than 5, or no more than 6 nucleotides from the central nucleotide(s) of the interrogatory hybridization region. In some embodiments, the interrogatory nucleotide is an internal nucleotide of the interrogatory probe. In some embodiments, the interrogatory probe does not comprise a terminal interrogatory nucleotide that has a free 3′ or 5′ terminus and is complementary to a corresponding nucleotide of interest in the variant. In some embodiments when the interrogatory hybridization region is at the 3′ end of the interrogatory probe, the interrogatory nucleotide is not the free 3′ terminal nucleotide of the interrogatory probe but can be any internal nucleotide residue, including the 5′ most nucleotide of the interrogatory hybridization region (which 5′ most nucleotide is linked to the rest of the interrogatory probe by a phosphodiester bond). In some embodiments, when the interrogatory hybridization region is at the 5′ end of the interrogatory probe, the interrogatory nucleotide is not the free 5′ terminal nucleotide of the interrogatory probe but can be any internal nucleotide residue, including the 3′ most nucleotide of the interrogatory hybridization region (which 3′ most nucleotide is linked to the rest of the interrogatory probe by a phosphodiester bond).

In some embodiments, the nucleotide at the free 5′ terminus of the interrogatory hybridization region is at position 1, and a single internal interrogatory nucleotide is at a nucleotide position between position 5 and position 11, inclusive, in the interrogatory hybridization region. In some embodiments, the nucleotide at the free 5′ terminus of the interrogatory hybridization region is at position 1, and a single internal interrogatory nucleotide is at a nucleotide position between position 3 and position 10, inclusive, in the interrogatory hybridization region. In some embodiments, the nucleotide at the free 5′ terminus of the interrogatory hybridization region is at position 1, and a single internal interrogatory nucleotide is at a nucleotide at position 3 in the interrogatory hybridization region. In some embodiments, the nucleotide at the free 5′ terminus of the interrogatory hybridization region is at position 1, and a single internal interrogatory nucleotide is at a nucleotide at position 5 in the interrogatory hybridization region. In some embodiments, the nucleotide at the free 5′ terminus of the interrogatory hybridization region is at position 1, and a single internal interrogatory nucleotide is at a nucleotide at position 9 in the interrogatory hybridization region. In some embodiments, the provided position of the internal interrogatory nucleotide is in an interrogatory probe with an interrogatory hybridization region that is 10 to 20 nucleotides in length. In some embodiments, the provided position of the internal interrogatory nucleotide is in an interrogatory probe with an interrogatory hybridization region that is 10 nucleotides in length. In some embodiments, the provided position of the internal interrogatory nucleotide is in an interrogatory probe with an interrogatory hybridization region that is 14 nucleotides in length. In some embodiments, the provided position of the internal interrogatory nucleotide is in an interrogatory probe with an interrogatory hybridization region that is 20 nucleotides in length.

In some embodiments, the interrogatory probe comprises one or more modified nucleotides or nucleotide analogs (e.g., LNA). In some embodiments, any one or more of the interrogatory nucleotides is a modified nucleotide or nucleotide analog, such as a locked nucleic acid (LNA) residue. In some embodiments, any one or more nucleotides adjacent to an interrogatory nucleotide is a modified nucleotide or nucleotide analog, such as a locked nucleic acid (LNA) residue. For example, an interrogatory probe, in some embodiments, comprises one or more modified nucleotide or nucleotide analog residues (e.g., LNA residue(s)) that are 5′ to an interrogatory nucleotide, and/or one or more modified nucleotide or nucleotide analog residues (e.g., LNA residue(s)) that are 3′ to the interrogatory nucleotide, in addition to the interrogatory nucleotide itself being a modified nucleotide or nucleotide analog such as LNA.

In some embodiments, the interrogatory hybridization region is about 2, about 5, about 10, about 15, about 20, about 25, about 30, about 35, about 40, about 45, about 50, or of any integer (or range of integers) of nucleotides in between the indicated values. In some embodiments, the interrogatory hybridization region is no more than about 40 nucleotides in length. In some embodiments, the interrogatory hybridization region is no more than about 15 nucleotides in length. In some embodiments, the interrogatory hybridization region is no more than about 10 nucleotides in length. In some embodiments, the interrogatory hybridization region is no more than about 30 nucleotides in length. In some embodiments, the interrogatory hybridization region is about 6, about 8, about 10, about 12, about 14, about 16, about 18, about 20, about 22, about 24, or of any integer (or range of integers) of nucleotides in between the indicated values. In some embodiments, the interrogatory hybridization region is about 10 nucleotides in length. In some embodiments, the interrogatory hybridization region is about 15 nucleotides in length. In some embodiments, the interrogatory hybridization region is about 20 nucleotides in length.

In some embodiments, the interrogatory hybridization region of the interrogatory probe is shorter than the constant hybridization region of the partner probe. In some embodiments, the interrogatory hybridization region of the interrogatory probe is shorter than the constant hybridization region of the partner probe by at least 5 nucleotides. In some embodiments, the interrogatory hybridization region of the interrogatory probe is shorter than the constant hybridization region of the partner probe by at least 10 nucleotides. In some embodiments, a short interrogatory hybridization region can lead to increased specificity of detecting short nucleotide sequences of interest (e.g., one or two nucleotides). In some embodiments, the interrogatory hybridization region is no more than about 15 nucleotides in length, and the constant hybridization region is more than about 15, more than about 20, or more than about 25 nucleotides in length. In some embodiments, the interrogatory hybridization region is no more than about 10 nucleotides in length, and the constant hybridization region is more than about 10, more than about 15, more than about 20, or more than about 25 nucleotides in length. In some embodiments, the interrogatory hybridization region is 10 nucleotides in length and the constant hybridization region is 20 nucleotides in length. In some embodiments, the interrogatory hybridization region is 14 nucleotides in length and the constant hybridization region is 19 nucleotides in length.

In some embodiments, the interrogatory hybridization region comprises a single internal interrogatory nucleotide complementary to a corresponding single nucleotide of interest in the variant. In some embodiments, the single nucleotide of interest is a single nucleotide variation (SNV), a single nucleotide polymorphism (SNP), a point mutation, a single nucleotide substitution, a single nucleotide insertion, or a single nucleotide deletion.

In some embodiments, the barcode region in the interrogatory probe may specifically correspond to the variant of the variable target sequence that the interrogatory probe targets. In some embodiments, the variant-specific barcode region is about 5, about 10, about 15, about 20, about 25, or about 30 nucleotides in length. The variant-specific barcode region may comprise one or more barcode sequences each of about 5, about 10, about 15, or about 20 nucleotides in length. In some embodiments, the variant-specific barcode region is about 10, about 20, about 30, about 40, about 50, about 60, about 70, or about 80 nucleotides in length. The variant-specific barcode region may comprise at least two, at least three, or at least four barcode sequences. In some embodiments, multiple barcode sequences in the barcode region are interrogated per interrogatory probe. In some embodiments, the readout of the nucleotide(s) of interest in the variable target sequence (e.g., SNP) is a combination of barcode sequences, and the combination of barcode sequences (instead of one variant-specific barcode sequence) is unique for the interrogating hybridization region in the same interrogatory probe.

In some embodiments, the splint hybridization region in the interrogatory probe is about 2, about 5, about 10, about 15, about 20, about 25, about 30, about 35, about 40, about 45, about 50, or of any integer (or range of integers) of nucleotides in between the indicated values. In some embodiments, the splint hybridization region is no more than about 40 nucleotides in length. In some embodiments, the splint hybridization region is no more than about 30 nucleotides in length. In some embodiments, the splint hybridization region is about 6, about 8, about 10, about 12, about 14, about 16, about 18, about 20, about 22, about 24, or of any integer (or range of integers) of nucleotides in between the indicated values. In some embodiments, the splint hybridization region is about 10 nucleotides in length.

In any of the embodiments herein, the splint oligonucleotide is between about 5 and about 50 nucleotides in length. In any of the embodiments herein, the splint hybridization region in the partner probe and the splint hybridization region in the interrogatory probe each are between about 5 and about 30 nucleotides in length. In any of the embodiments herein, the splint oligonucleotide is between about 10 and about 25 nucleotides in length.

In some embodiments, the interrogatory probe is about 30, about 40, about 50, about 60, about 70, about 80, about 90, about 100, about 110, about 120, about 130, about 140, about 150, about 160, about 170, about 180, about 190, about 200 or more nucleotides in length.

In some embodiments, the variable target sequence and the constant target sequence are linked by one phosphodiester bond. In some embodiments, the variable target sequence and the constant target sequence are linked by one or more nucleotide residues.

In some embodiments, the constant target sequence is about or at least 4, about or at least 6, about or at least 8, about or at least 10, about or at least 12, about or at least 14, about or at least 16, about or at least 18, about or at least 20, or more nucleotides in length. In some embodiments, the constant target sequence is common among multiple molecules of the first target nucleic acid that comprise different variants of the variable target sequence.

In some embodiments, the constant hybridization region is about 2, about 5, about 10, about 15, about 20, about 25, about 30, about 35, about 40, about 45, about 50, or of any integer (or range of integers) of nucleotides in between the indicated values. In some embodiments, the constant hybridization region is no more than about 40 nucleotides in length. In some embodiments, the constant hybridization region is no more than about 30 nucleotides in length. In some embodiments, the constant hybridization region is about 6, about 8, about 10, about 12, about 14, about 16, about 18, about 20, about 22, about 24, or of any integer (or range of integers) of nucleotides in between the indicated values. In some embodiments, the constant hybridization region is about 20 nucleotides in length.

In some embodiments, the barcode region (if present) in the partner probe may specifically correspond to the first target nucleic acid (or a sequence thereof) that the partner probe targets, but may not specifically correspond to any one or more different variants of the variable target sequence of the first target nucleic acid. In some embodiments, the barcode region in the partner probe is a gene-specific barcode region. In some embodiments, the gene-specific barcode region is about 5, about 10, about 15, about 20, about 25, or about 30 nucleotides in length. The gene-specific barcode region may comprise one or more barcode sequences each of about 5, about 10, about 15, or about 20 nucleotides in length.

In some embodiments, the partner probe does not comprise any barcode region and may instead comprise a common or universal spacer region. In some embodiments, the partner probe does not any barcode region or spacer region between the constant hybridization region and the splint hybridization region of the partner probe. In some embodiments, a partner probe disclosed herein does not comprise any nucleic acid barcode sequence. In some embodiments, partner probes for hybridizing to multiple different target nucleic acids comprise a common sequence that is not complementary to the target nucleic acids. For instance, the backbone sequences of a plurality of partner probes for detecting different variant sequences of a target nucleic acid can be a common backbone sequence. In other examples, the backbone sequences of a plurality of partner probes for detecting different target nucleic acids comprise a common backbone sequence, and the arms of the partner probes can be different such that they specifically hybridize to the different target nucleic acids. In some embodiments, the backbone sequences of the plurality of partner probes do not contain any nucleic acid barcode sequence that uniquely corresponds to a particular target nucleic acid or a particular sequence variant thereof.

In some embodiments, the splint hybridization region in the partner probe is about 2, about 5, about 10, about 15, about 20, about 25, about 30, about 35, about 40, about 45, about 50, or of any integer (or range of integers) of nucleotides in between the indicated values. In some embodiments, the splint hybridization region is no more than about 40 nucleotides in length. In some embodiments, the splint hybridization region is no more than about 30 nucleotides in length. In some embodiments, the splint hybridization region is about 6, about 8, about 10, about 12, about 14, about 16, about 18, about 20, about 22, about 24, or of any integer (or range of integers) of nucleotides in between the indicated values. In some embodiments, the splint hybridization region is about 10 nucleotides in length.

In some embodiments, the partner probe is about 30, about 40, about 50, about 60, about 70, about 80, about 90, about 100, about 110, about 120, about 130, about 140, about 150, about 160, about 170, about 180, about 190, about 200 or more nucleotides in length.

In some embodiments, the circularized probe formed by the interrogatory probe and the partner probe is about 50, about 60, about 70, about 80, about 90, about 100, about 110, about 120, about 130, about 140, about 150, about 160, about 170, about 180, about 190, about 200 or more nucleotides in length.

In some embodiments, the interrogatory probe and the partner probe in the same probe pair targeting the first target nucleic acid are of the same length. In some embodiments, the interrogatory probe and the partner probe are symmetric. For instance, the interrogatory probe can comprise from 5′ to 3′: an interrogatory hybridization region of about 20 nucleotides, a variant-specific barcode region of about 16 nucleotides, and a splint hybridization of about 10 nucleotides; and the partner probe can comprise from 5′ to 3′: a splint hybridization of about 10 nucleotides, a gene-specific barcode region of about 16 nucleotides, and a constant hybridization region of about 20 nucleotides. In other examples, the interrogatory probe comprises from 3′ to 5′: an interrogatory hybridization region of about 20 nucleotides, a variant-specific barcode region of about 16 nucleotides, and a splint hybridization of about 10 nucleotides; and the partner probe comprises from 3′ to 5′: a splint hybridization of about 10 nucleotides, a gene-specific barcode region of about 16 nucleotides, and a constant hybridization region of about 20 nucleotides.

In some embodiments, the interrogatory probe and the partner probe in the same probe pair targeting the first target nucleic acid are of different lengths. In some embodiments, the interrogatory probe and the partner probe are asymmetric. The interrogatory probe can be longer or shorter than the partner probe. For instance, the interrogatory probe can comprise from 5′ to 3′: an interrogatory hybridization region of about 20 nucleotides, a variant-specific barcode region of about 16 nucleotides, and a splint hybridization of about 7 nucleotides; and the partner probe can comprise from 5′ to 3′: a splint hybridization of about 13 nucleotides, a spacer region of about 5 nucleotides, and a constant hybridization region of about 20 nucleotides. In other examples, the interrogatory probe comprises from 3′ to 5′: an interrogatory hybridization region of about 20 nucleotides, a variant-specific barcode region of about 16 nucleotides, and a splint hybridization of about 7 nucleotides; and the partner probe comprises from 3′ to 5′: a splint hybridization of about 13 nucleotides, a spacer region of about 5 nucleotides, and a constant hybridization region of about 20 nucleotides.

In some embodiments, the interrogatory hybridization region of the interrogatory probe is shorter than the constant hybridization region of the partner probe. In some embodiments, the interrogatory region is between about 5 and about 15 nucleotides in length, between 5 and about 12 nucleotides in length, between about 5 and about 10 nucleotides in length, or between about 5 and about 8 nucleotides in length. In some embodiments, the constant hybridization region is between about 15 and about 40 nucleotides in length, between about 15 and about 35 nucleotides in length, between about 15 and about 30 nucleotides in length, or between about 15 and about 25 nucleotides in length. In some embodiments, the interrogatory hybridization region comprises a single internal interrogatory nucleotide complementary to a corresponding single nucleotide of interest in the variant, wherein the internal interrogatory nucleotide is not at a free 3′ end or 5′ end of the interrogatory hybridization region.

In some embodiments, the partner probe and the interrogatory probe each comprise a splint hybridization region complementary to a splint oligonucleotide, and upon hybridization to the splint oligonucleotide, the splint hybridization regions are configured to be connected, e.g., ligated using the splint oligonucleotide as a template, with or without gap filling and/or cleavage of a 5′ flap prior to the ligation. In some embodiments, the splint oligonucleotide is between about 20 and about 25 nucleotides in length.

In some embodiments, the interrogatory probe and/or the partner probe for the first target nucleic acid and/or the circularizable probe for the second target nucleic acid each independently comprises one or more ribonucleotide residues at and/or near a 3′ end. In some embodiments, the one or more ribonucleotide residues are ligatable ribonucleotide residues.

In some embodiments, the partner probe and the interrogatory probe are ligated using the first target nucleic acid as a template, with or without gap filling and/or cleavage of a 5′ flap prior to the ligation. In some embodiments, the first target nucleic acid is an RNA or a DNA, such as an mRNA, cDNA, or genomic DNA.

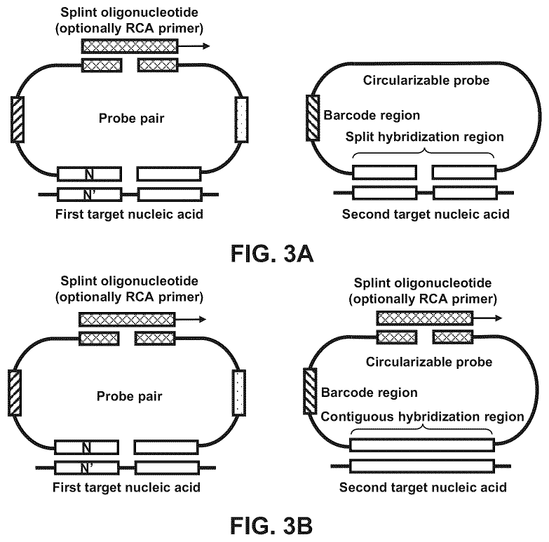

In some embodiments, provided herein is a method for analyzing a biological sample, the method comprising contacting the biological sample with a circularizable probe comprising a first probe region and a second probe region (e.g., 5′ and 3′ arms of a padlock probe) that hybridize to a second target nucleic acid (e.g., an RNA or cDNA) in the biological sample. In some embodiments, the first probe region and the second probe region form a split hybridization region, e.g., as shown in FIG. 3A, and the first and second probe regions hybridize to a first target sequence and a second target sequence, respectively, in the second target nucleic acid. The first and second probe regions can be at the 5′ and 3′ ends of circularizable probe or vice versa.

In some examples, the second target nucleic acid comprises, from 5′ to 3′: the first target sequence and the second target sequence. In some embodiments, the circularizable probe comprises, from 5′ to 3′: the first probe region complementary to the first target sequence; a barcode region corresponding to the second target nucleic acid or a sequence thereof; and the second probe region complementary to the second target sequence. In some examples, the second target nucleic acid comprises, from 5′ to 3′: the second target sequence and the first target sequence. In some embodiments, the circularizable probe comprises, from 5′ to 3′: the second probe region complementary to the second target sequence; a barcode region corresponding to the second target nucleic acid or a sequence thereof; and the first probe region complementary to the first target sequence. In some embodiments, the circularizable probe comprises, from 5′ to 3′: the first probe region complementary to the first target sequence; a barcode region corresponding to the second target nucleic acid or a sequence thereof; an anchor region; and the second probe region complementary to the second target sequence. For example, the anchor region comprises a sequence that is common among circularizable probes for two or more different target nucleic acids. In some embodiments, the first probe region and the second probe region do not comprise an interrogatory nucleotide that is configured to base pair with a target nucleotide (a nucleotide of interest) in the first target sequence or the second target sequence. In some embodiments, the anchor region of the circularizable probe is used to hybridize a primer for rolling circle amplification.

In some embodiments, upon hybridization to the second target nucleic acid, the 5′ and 3′ target hybridization regions of the circularizable probe is configured to be ligated using the second target nucleic acid as a template, with or without gap filling and/or cleavage of a 5′ flap prior to the ligation. In some embodiments, ligation of the circularizable probe hybridized to the second target nucleic acid forms a second circularized probe.

In some embodiments, the first probe region and the second probe region of the circularizable probe forms a contiguous hybridization region, e.g., as shown in FIG. 3B, and hybridize to a contiguous target sequence in the second target nucleic acid. In some embodiments, the circularizable probe comprises splint hybridization regions that are complementary to a splint. In some examples, the circularizable probe comprises, from 5′ to 3′: a 5′ splint hybridization region (e.g., a splint hybridization region having a ligatable 5′ terminal nucleotide residue); a barcode region corresponding to the second target nucleic acid or a sequence thereof; a contiguous hybridization region complementary to the second target nucleic acid; and a 3′ splint hybridization region (e.g., a splint hybridization region having a ligatable 3′ terminal nucleotide residue). In some examples, the circularizable probe comprises, from 5′ to 3′: a 5′ splint hybridization region (e.g., a splint hybridization region having a ligatable 5′ terminal nucleotide residue); a contiguous hybridization region complementary to the second target nucleic acid; a barcode region corresponding to the second target nucleic acid or a sequence thereof, and a 3′ splint hybridization region (e.g., a splint hybridization region having a ligatable 3′ terminal nucleotide residue).

In some embodiments, the 5′ and 3′ splint hybridization regions of the circularizable probe are complementary to a splint oligonucleotide, and upon hybridization to the splint oligonucleotide, the 5′ and 3′ splint hybridization regions are configured to be ligated using the splint oligonucleotide as a template, with or without gap filling and/or cleavage of a 5′ flap prior to the ligation.

In some embodiments, the circularizable probe comprises: a 5′ target hybridization region, a barcode region corresponding to the second target nucleic acid, and a 3′ target hybridization region, and upon hybridization to the second target nucleic acid, the 5′ and 3′ target hybridization regions are configured to be ligated using the second target nucleic acid as a template, with or without gap filling and/or cleavage of a 5′ flap prior to the ligation.

The circularizable probe for the second target nucleic acid may but does not need to comprise a barcode region corresponding to the second target nucleic acid or a sequence thereof. In some embodiments, a sequence that is complementary to the second target nucleic acid, for instance, a sequence in the first probe region and/or the second probe region, or in the contiguous hybridization region, is detected as an identifier sequence for the second target nucleic acid. In some embodiments, the barcode sequence of the circularizable probe is specific to the second target nucleic acid but not specific to any one or more variants of the second target nucleic acid. For instance, an RCP of the circularizable probe can be generated and the RCP comprises multiple copies of a sequence in the target sequence (which is targeted by and complementary to the circularizable probe) in the second target nucleic acid, and the multiple copies of the sequence in the RCP can be detected as an identifier sequence in order to detect the second target nucleic acid.

In some embodiments, an interrogatory probe and/or a partner probe (targeting a first target nucleic acid) and/or a circularizable probe (targeting a second target nucleic acid) disclosed herein can each independently comprise one or more barcode sequences. A barcode sequence, if present in a probe, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of nucleic acid probes is less than the number of distinct targets of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes. In some embodiments, the barcode sequences or any subset thereof in the population of nucleic acid probes are independently and/or combinatorially detected and/or decoded.

In some embodiments, a probe disclosed herein (e.g., an interrogatory probe, a partner probe, or a circularizable probe provided as a single molecule) comprises a 5′ flap which may be recognized by a structure-specific cleavage enzyme, e.g. an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include flap endonucleases (FENS), which are a class of enzymes having endonucleolytic activity and being capable of catalyzing the hydrolytic cleavage of the phosphodiester bond at the junction of single- and double-stranded DNA. Thus, in some embodiment, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693-24700 and in US 2020/0224244 and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. 1999. PNAS 96, 6143-6148 for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

In some embodiments, any of the ligation reactions disclosed herein can involve template dependent ligation, e.g., using the first target nucleic acid, the second target nucleic acid, and/or the splint oligonucleotide as template. In some embodiments, the ligation can involve template independent ligation. In some embodiments, any of the ligation reactions disclosed herein can involve chemical ligation. In some embodiments, any of the ligation reactions disclosed herein can involve click chemistry.

In some embodiments, any of the ligation reactions disclosed herein can involve enzymatic ligation. In some embodiments, the enzymatic ligation involves use of a ligase. In some aspects, the ligase used herein comprises an enzyme that is commonly used to join polynucleotides together or to join the ends of a single polynucleotide. In some aspects, the ligase used herein is a DNA ligase. In some aspects, the ligase used herein is an ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity. In some embodiments, the ligase is a ssDNA ligase. In some embodiments, the ssDNA ligase is a bacteriophage TS2126 RNA ligase or an archaebacterium RNA ligase or a variant or derivative thereof. In some embodiments, the ligase is Methanobacterium thermoautotrophicum RNA ligase 1, CircLigase™ I, CircLigase™ II, T4 RNA ligase 1, or T4 RNA ligase 2, or a variant or derivative thereof.

In some embodiments, to form the first circularized probe, the ligation of the partner probe and the interrogatory probe templated on the first target nucleic acid and the ligation of the two probes templated on the splint oligonucleotide are performed using the same ligase. In some embodiments, the ligation of the partner probe and the interrogatory probe (e.g., ligation templated on the first target nucleic acid) and the ligation of the circularizable probe templated on the second target nucleic acid are performed using the same ligase. In some embodiments, the ligase can have an RNA-templated ligase activity and/or a DNA-templated ligase activity. In some embodiments, both the first and second target nucleic acids are RNA and a ligase is used to ligate adjacent, single-stranded DNA (e.g., in probe molecules) splinted by a complementary RNA strand (e.g., the target RNA). In some embodiments, a pair of interrogatory probe and partner probe is provided for each different variant of the first target nucleic acid, and the splint hybridization regions in the probe pairs for two or more different variants of the first target nucleic acid are complementary to a common splint oligonucleotide (e.g., a common anchor splint). In some embodiments, the splint oligonucleotide that is common for multiple different probe pairs is used as a primer for RCA.

In some embodiments, the ligation of the partner probe and the interrogatory probe and the ligation of the circularizable probe is performed using two or more different ligases in the same ligation step or in different ligation steps. In some embodiments, the different ligation steps are performed consecutively, e.g., each using an RNA-templated ligase or a DNA-templated ligase. In some embodiments, the ligation templated on a target RNA (e.g., the first target nucleic acid and/or the second target nucleic acid) is performed using a first ligase having an RNA-templated ligase activity. In some embodiments, the ligation templated on a splint oligonucleotide (e.g., a DNA splint) is performed using a second ligase having a DNA-templated ligase activity. The first and second ligases can be contacted with the biological sample consecutively or simultaneously. For instance, the first and second ligases can be pre-mixed prior to contacting the sample, or added to the sample at the same time in separate compositions. The RNA-templated ligation can be performed before, simultaneously with, or after the DNA-templated ligation.

In some aspects, a high fidelity ligase, such as a thermostable DNA ligase (e.g., a Taq DNA ligase), is used, for example, for ligation templated on a splint oligonucleotide to ligate an interrogatory probe to a partner probe or to circularize a circularizable probe disclosed herein. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the interrogatory hybridization region comprises a sequence complementary to a nucleotide variation, a nucleotide polymorphism, a mutation, a substitution, an insertion, a deletion, a translocation, a duplication, an inversion, and/or a repetitive sequence, for identifying a variant sequence among a plurality of different sequences in situ in a biological sample. In some embodiments, the interrogatory hybridization region comprises a sequence complementary to a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the interrogatory hybridization region comprises a sequence complementary to a sequence comprising multiple nucleotides, and each nucleotide is independently at the position of an SNV, an SNP, a point mutation, a single nucleotide substitution, a single nucleotide insertion, or a single nucleotide deletion. In some embodiments, the constant hybridization region of a partner probe is complementary to a common first exon (e.g., Exon 1 in FIG. 2C) and different interrogatory probes comprise hybridization regions complementary to different second exons (e.g., Exon 1 and Exon 2 in FIG. 2C), and probe pairs are used to analyze splice junctions and isoforms of splicing.

In some embodiments, provided herein is a library of interrogatory probes comprising i) an interrogatory probe comprising a sequence in its interrogatory hybridization region that is complementary to a nucleotide variation, a nucleotide polymorphism, a mutation, a substitution, an insertion, a deletion, a translocation, a duplication, an inversion, and/or a repetitive sequence, and ii) another interrogatory probe which does not comprise a sequence complementary to the nucleotide variation, nucleotide polymorphism, mutation, substitution, insertion, deletion, translocation, duplication, inversion, and/or repetitive sequence. In some embodiments, the library of interrogatory probe comprises i) an interrogatory probe comprising a sequence in its interrogatory hybridization region that is complementary to a variant sequence or deletion or insertion, and ii) another interrogatory probe which does not comprise a sequence complementary to the variant sequence or deletion or insertion. For example, wildtype and variant interrogatory probes in the library, when contacted with the biological sample, can compete with one another for hybridization to a variable target sequence comprising a variant sequence, and the matching interrogatory probe can outcompete other interrogatory probes which do not match the variant sequence (e.g., one or more nucleotides) in the variable target sequence. The competition among interrogatory probes can allow the use of short interrogatory hybridization regions in the interrogatory probes, while achieving specificity of interrogatory probe/partner probe hybridization and/or ligation, for instance, when probe hybridization and ligation are performed in the same reaction mix and/or the same reaction condition. In some embodiments, using a low hybridization temperature, less denaturation, and/or more co-factors such as Mg²⁺ or other factors that promote hybridization can enable the use of shorter interrogatory hybridization regions in the interrogatory probes.

In some embodiments, the partner probe is hybridized to the target nucleic acid, followed by contacting the biological sample with a library of interrogatory probes that compete for hybridization to the target nucleic acid (e.g., hybridization to the variable target sequence in the target nucleic acid). In some embodiments, the hybridization of an interrogatory probe to the target nucleic acid and the ligation of the interrogatory probe to the partner probe are performed sequentially, e.g., the interrogatory probe hybridization is performed in a reaction condition or reaction mix, and the interrogatory probe ligation to the partner probe is performed in a different reaction condition or different reaction mix. In some embodiments, the hybridization of an interrogatory probe to the target nucleic acid and the ligation of the interrogatory probe to the partner probe are performed in the same reaction condition or the same reaction mix.

In some embodiments, the partner probe and the library of interrogatory probes are contacted with the target nucleic acid at the same time, in the same reaction mix or separately. For example, in some embodiments, the partner probe and the library of interrogatory probes are premixed before contacting the biological sample with the mixture. In another example, two separate compositions comprising the partner probe and the library of interrogatory probes, respectively, are contacted with the biological sample. In some embodiments, the hybridization of an interrogatory probe to the target nucleic acid and the ligation of the interrogatory probe to the partner probe are performed in the same reaction condition or the same reaction mix.

In some embodiments, the probes targeting the first target nucleic acid (e.g., the partner probe and the interrogatory probe) and the circularizable probe targeting the second target nucleic acid are contacted with the biological sample at the same time, in the same reaction mix or separately, or sequentially in any order. In some embodiments, the probes targeting the first target nucleic acid (e.g., the partner probe and the interrogatory probe) and the circularizable probe targeting the second target nucleic acid are contacted with the biological sample at the same time and in the same reaction mix. In some embodiments, the probes targeting the first target nucleic acid (e.g., the partner probe and the interrogatory probe) and a panel of circularizable probes targeting a plurality of different target nucleic acids (e.g., different transcripts) are contacted with the biological sample at the same time.

In some embodiments, the biological sample is contacted with a library of interrogatory probes each: i) targeting a different variant of the variable target sequence of the first target nucleic acid, and ii) comprising a variant-specific barcode region. In some embodiments, the biological sample is contacted with multiple partner probes. In some embodiments, the multiple partner probes are identical such that a common partner probe is used in conjunction with the different interrogatory probes. In some embodiments, the multiple partner probes share the same constant hybridization region (such that they all target the first target nucleic acid) but they may comprise different sequences outside the common constant hybridization region. For instance, two or more partner probes can each comprise a different barcode region, a different spacer region, and/or a different splint hybridization region. In some embodiments, the library of interrogatory probes and/or the multiple partner probes independently comprise at least about 2, at least about 4, at least about 10, at least about 20, at least about 50, at least about 100, or more oligonucleotides of different sequences. In some embodiments, the sequence diversity of the interrogatory hybridization regions in the library is such that at least or about 80%, at least or about 85%, at least or about 90%, at least or about 95%, or about 100% of the possible variant sequences in the variable target sequence of the first target nucleic acid in a sample have corresponding interrogatory probes in the library.

In some embodiments, the biological sample is contacted with the probes under conditions permissive for specific hybridization of an interrogatory probe to its complementary sequence in the variable target sequence (and specific hybridization of a partner probe to the constant target sequence) in the first target nucleic acid, and/or specific hybridization of a circularizable probe to the second target nucleic acid. In some embodiments, the circularized probes are amplified by RCA (e.g., as described in Section IV). In some embodiments, a sequence in an RCA product is determined in situ, e.g., by sequencing the barcode sequences using SBS, SBB, SBH, SBL, or sequential hybridization of probes, etc., as described in Section V.

### IV. Rolling Circle Amplification (RCA)

Following formation of the circularized probe, in some instances, a primer oligonucleotide is added for amplification. In some instances, the primer oligonucleotide is added with the interrogatory probe, the partner probe, and/or the circularizable probe. In some instances, the primer oligonucleotide is added before or after the interrogatory probe, the partner probe, and/or the circularizable probe is contacted with the sample. In some instances, the primer oligonucleotide for amplification of the circularized probe may comprise a sequence complementary to a target nucleic acid, as well as a sequence complementary to the circularized probe that hybridizes to the target nucleic acid. In some embodiments, a washing step is performed to remove any unbound probes, primers, etc. In some embodiments, the wash is a stringency wash. In some embodiments, a plurality of washes are performed after probe hybridization. In some embodiments, the one or more post probe hybridization wash is performed with a buffer that is suitable for a following ligation reaction. In some embodiments, the wash is performed with a wash buffer comprising potassium chloride (KCl) and polyethylene glycol (PEG). In some embodiments, the wash is performed with a wash buffer comprising potassium chloride (KCl). In some embodiments, the wash is performed with a wash buffer comprising polyethylene glycol (e.g., 15% PEG800). Washing steps can be performed at any point during the process to remove non-specifically bound probes, probes that have ligated, etc.

In some embodiments, a splint oligonucleotide used to ligate the interrogatory probe, the partner probe, and/or the circularizable probe, or a portion of the splint oligonucleotide, is used to prime the RCA of a circularized probe. For instance, the splint oligonucleotide can be cleaved and a portion that remains hybridized to the circularized probe can be used as an RCA primer. A separate primer oligonucleotide may but does not need to be used for the RCA. In some embodiments, the target nucleic acid (or a portion thereof) hybridized to the ligated interrogatory probe and the partner probe is used to prime the RCA of the circularized probe.

In some embodiments, the first target nucleic acid, the second target nucleic acid, or a portion of the first or second target nucleic acid that is hybridized to a circularized probe, is used to prime the RCA of the circularized probe. A separate primer oligonucleotide may but does not need to be used for the RCA. For instance, the first or second target nucleic acid can be RNA and can be cleaved (e.g., via RNase H digestion) and a portion that remains hybridized to the circularized probe can be used as an RCA primer. In some embodiments, amplification of the circularized probe (e.g., formed from the ligated interrogatory probe and the partner probe binding a first target nucleic acid, and/or the circularizable probe binding a second target nucleic acid) is primed by the respective target RNAs. In some embodiments, the target RNAs are immobilized in the biological sample. In some embodiments, the target RNAs is cleaved by an enzyme (e.g., RNase H). In some embodiments, the target RNA is cleaved at a position downstream of the target sequences bound to the circularized probe. In some aspects, the methods disclosed herein allow targeting of RNase H activity to a particular region in a target RNA that is adjacent to or overlapping with a target sequence for a probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe by about 1 to about 20 nucleotides or by about 8 to about 10 nucleotides. In some embodiments, the cleaved target RNA itself is then used to prime RCA of the circularized probe. In some cases, a plurality of nucleic acid oligonucleotides are used to perform target-primed RCA for a plurality of different target RNAs.

In any of the embodiments herein, the biological sample is contacted with the RNase H (and optionally with the nucleic acid oligonucleotide) before or during formation of the circularized probe. In some embodiments, the biological sample is contacted with the oligonucleotide and with the RNase H simultaneously or sequentially (in either order) before contacting the sample with the probes. In any of the embodiments herein, the biological sample is contacted with the RNase H (and optionally with the nucleic acid oligonucleotide) after formation of the circularized probe(s). In any of the embodiments herein, the RNase H comprises an RNase H1 and/or an RNAse H2. In some embodiments, RNase inactivating agents or inhibitors are added to the sample after cleaving the target RNA.

The splint oligonucleotide used for probe ligation/circularization or a portion thereof may but does not need to be used for the RCA. For instance, as shown in FIGS. 3A-3B, the splint oligonucleotide used to ligate the pair of interrogatory probe and partner probe and the splint oligonucleotide used to ligate the circularizable probe can be the same or different in sequence, and either or both of the splint oligonucleotides can be used to primer RCA of the respective circularized probe. Likewise, a separate primer oligonucleotide may but does not need to be used for the RCA. In some embodiments, a primer that hybridizes to the anchor region of the circularized probe hybridized to the second target nucleic acid is used to prime the RCA of the circularized probe.

In some embodiments, a primer oligonucleotide for amplification of the circularized probe comprises a single-stranded nucleic acid sequence having a 3′ end that can be used as a substrate for a nucleic acid polymerase in a nucleic acid extension reaction. The primer oligonucleotide can comprise both RNA nucleotides and DNA nucleotides (e.g., in a random or designed pattern). The primer oligonucleotide can also comprise other natural or synthetic nucleotides described herein that can have additional functionality. In some embodiments, the primer oligonucleotide is about 6 bases to about 100 bases, such as about 25 bases.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of, e.g., a circularizable probe), the circularized probe is rolling-circle amplified to generate a RCA product (e.g., amplicon) containing multiple copies of the sequence of the circularized probe.

In some embodiments, RCPs are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is Phi29 DNA polymerase.

In some embodiments, the polymerase comprises a modified recombinant Phi29-type polymerase. In some embodiments, the polymerase comprises a modified recombinant Phi29, B103, GA-1, PZA, Phi15, BS32, M2Y, Nf, G1, Cp-1, PRD1, PZE, SF5, Cp-5, Cp-7, PR4, PR5, PR722, or L17 polymerase. In some embodiments, the polymerase comprises a modified recombinant DNA polymerase having at least one amino acid substitution or combination of substitutions as compared to a wildtype Phi29 polymerase. Exemplary polymerases are described in U.S. Pat. Nos. 8,257,954; 8,133,672; 8,343,746; 8,658,365; 8,921,086; and 9,279,155, all of which are herein incorporated by reference. In some embodiments, the polymerase is not directly or indirectly immobilized to a substrate, such as a bead or planar substrate (e.g., glass slide), prior to contacting a sample, although the sample may be immobilized on a substrate.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some aspects, during the amplification step, modified nucleotides are added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide reacts with an acrylic acid N-hydroxysuccinimide moiety. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification. In some embodiments, the modified nucleotides comprises base modifications, such as azide and/or alkyne base modifications, dibenzylcyclooctyl (DBCO) modifications, vinyl modifications, trans-Cyclooctene (TCO), and so on.

In some embodiments, the primer extension reaction mixture comprises a deoxynucleoside triphosphate (dNTP) or derivative, variant, or analogue thereof. In some embodiments, the primer extension reaction mixture comprises a catalytic cofactor of the polymerase. In some embodiments, the primer extension reaction mixture comprises a catalytic di-cation, such as Mg²⁺ and/or Mn²⁺.

In some aspects, the amplification product (e.g., RCA product) can be anchored to a polymer matrix. The amplification products may be immobilized within the matrix generally at the location of the nucleic acid being amplified. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products (e.g., RCA products) are copolymerized and/or covalently attached to the surrounding matrix thereby preserving their spatial relationship and any information inherent thereto. In some embodiments, the RCA products are generated from DNA or RNA within a cell embedded in the matrix. In some embodiments, the RCA products can also be functionalized to form covalent attachment to the matrix preserving their spatial information within the cell thereby providing a subcellular localization distribution pattern. In some embodiments, the provided methods involve embedding RCA products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing or probe hybridization while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

### V. Detection and Analysis

In some aspects, the provided methods involve analyzing, e.g., detecting or determining, one or more nucleic acid sequences such as variable target sequences in target nucleic acids. In some cases, the analysis is performed on one or more images captured, and may comprise processing the image(s) and/or quantifying signals observed. In some embodiments, the analysis comprises detecting a sequence (e.g., a variable target sequence) present in the sample. In some embodiments, the analysis comprises quantification of puncta (e.g., if amplification products are detected). In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further comprises displaying the information from the analysis or detection step. In some embodiments, software may be used to automate the processing, analysis, and/or display of data.

In some embodiments, following amplification, the sequence of the amplicon (e.g., RCA product) or a portion thereof, is determined or otherwise analyzed, for example by using detectably labeled probes and imaging. The analysis of the amplification products can comprise sequencing-by-synthesis (SBS), sequencing-by-binding (SBB), avidity sequencing, sequencing-by-ligation (SBL), sequencing-by-hybridization (SBH), and/or fluorescent in situ hybridization, and/or wherein the in situ hybridization comprises sequential hybridization of probes.

In some embodiments where SBS or SBB is used to determine a sequence (e.g., barcode region) in the RCA product, the biological sample is contacted with nucleotides in sequential cycles, where in each cycle a complex is formed, the complex comprising i) the sequencing primer or an extension product thereof hybridized to the sequencing primer binding site 3′ to the barcode sequence, ii) a polymerase, and iii) a cognate nucleotide that base pairs with a nucleotide in the variable target sequence, and a signal (ON) and/or an absence of signal (OFF) associated with the cognate nucleotide and/or the polymerase in the complex is detected at a particular location in the biological sample, wherein the ON signal, the OFF signal, or a combination thereof corresponds to the base in the cognate nucleotide and the corresponding nucleotide in the barcode sequence. In some embodiments, a signal code corresponding to the ON signal, the OFF signal, or a combination thereof is detected at the particular location. In some embodiments, the signal code corresponds to a signal of a first color, a signal of a second color, a signal of a third color, or absence of signal, wherein the first, second, and third colors are different. In some embodiments, the signal code corresponds a combination of signals of a first or second color, or absence of signal, wherein the first and second colors are different. In some embodiments, the signal code corresponds to a combination of ON and/or OFF signals, wherein the combination of ON and/or OFF signals is detected in two or more imaging steps.

In some embodiments, a barcode sequence (e.g., in the RCP) is detected by SBB using a polymerase that is fluorescently labeled and one or more nucleotides that are not fluorescently labeled. In some embodiments, during SBB, a cognate nucleotide is not incorporated by the polymerase into the sequencing primer or an extension product thereof. In some embodiments, incorporation of a cognate nucleotide by the polymerase into the sequencing primer or an extension product thereof is attenuated or inhibited. Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, a barcode sequence (e.g., in the RCP) is detected by SBS, comprising contacting the biological sample with a nucleotide mix comprising a fluorescently labeled nucleotide and a nucleotide that is not fluorescently labeled. In some embodiments, during SBS, a cognate nucleotide is incorporated by the polymerase into the sequencing primer or an extension product thereof, and the cognate nucleotide may or may not be fluorescently labeled.

In some embodiments where SBL is used, the biological sample is contacted with an anchor of known sequence and detectably labeled probes, one of which are complementary to a sequence in the RCA product. The anchor can be 3′ or 5′ to the barcode sequence to be determined. In some embodiments, after hybridization of the complementary detectably labeled probe to the barcode sequence, the detectably labeled probe is ligated to the anchor or an extended product thereof, whereas detectably labeled probes that are not complementary to the barcode sequence are not ligated and can be removed, e.g., by washing the sample. In some embodiments, signals associated with the complementary detectably labeled probe ligated to the anchor or extension product thereof are detected, thereby detecting the corresponding sequence in the barcode region.

In some embodiments, the detection or determination comprises hybridizing one or more probes to nucleic acid molecules such as RCPs (e.g., described in Section IV and Section V). In some embodiments, the in situ detection herein comprises sequential hybridization of probes, e.g., sequencing by hybridization and/or sequential in situ fluorescence hybridization. Sequential fluorescence hybridization can involve sequential hybridization of the intermediate probes and detectably labeled probes disclosed herein. In some embodiments, a method disclosed herein comprises sequential hybridization of detectably labeled probes and intermediate probes that are not detectably labeled per se but are capable of binding (e.g., via nucleic acid hybridization) and being detected by detectably labeled probes, such as fluorescently labeled probes. Exemplary methods comprising sequential fluorescence hybridization of probes are described in US 2019/0161796, US 2020/0224244, US 2022/0010358, US 2021/0340618, US 2022/0064697, and US 2023/0039899, all of which are incorporated herein by reference.

In some embodiments, the detection or determination comprises detecting one or more barcode sequences associated with the variable target sequence in a temporally sequential manner for in situ analysis in a biological sample, e.g., in an intact tissue. In some aspects, provided herein is a method for detecting the detectably labeled probes, thereby generating a signal code. In some instances, each signal code corresponds to a sequence variant of a variable target sequence. In some instances, one signal code corresponds to a wildtype sequence comprised by a variable target sequence while a different signal code corresponds to any mutated sequence compared to the wildtype sequence. In some instances, the probes may be optically detected (e.g., by detectably labeled probes) in a temporally-sequential manner. In some embodiments, the sample is contacted with a library of probes to detect the probes or products thereof (e.g., used or generated as described in Sections III and IV, such as RCPs) associated with the variable target sequence in each target nucleic acid. For example, each one or more variants of a variable target sequence in a first target nucleic acid can be assigned a different signal code sequence which corresponds to a barcode region in an interrogatory probe of a library of interrogatory probes targeting the first target nucleic acid. Likewise, each one or more other target nucleic acids (e.g., a second target nucleic acid, a third target nucleic acid, etc.) can be assigned a different signal code sequence which corresponds to a barcode region in circularizable probe(s) targeting the one or more other target nucleic acids. In some embodiments, the barcode regions for the variants of the first target nucleic acid and the barcode regions for the one or more other target nucleic acids are decoded in the same decoding scheme, using probe hybridization in sequential cycles to detect the signal code sequences at locations in a biological sample, thereby not only detecting the target nucleic acids (the first target nucleic acid, the second target nucleic acid, the third target nucleic acid, etc.) but also the variants of the first target nucleic acid in situ in the biological sample.

In some aspects, the method comprises sequential hybridization of labelled probes to create a signal code sequence (e.g., a temporal pattern of signal codes corresponding to signals detected at a location) that identifies the variable target sequence or portion thereof. In some instances, each interrogatory hybridization region and the barcode region in the same interrogatory probe is associated with a different signal code sequence. In some instances, the interrogatory hybridization region corresponding to the wildtype sequence is associated with a signal code sequence, and the interrogatory hybridization regions corresponding to all mutated sequences are associated with a different signal code sequence that is shared among the mutated sequences. In such instances, multiple cycles of probe hybridization and detection are performed to detect multiple sequence variants (e.g., more than three, four, five, six, seven, eight, or more, including wildtype sequence) at location(s) in a biological sample.

In some embodiments, provided herein is a method of analyzing a sample, comprising: a) producing an RCA product in the sample, the RCA product comprising multiple copies of a complementary barcode sequence, wherein the complementary barcode sequence (and the barcode sequence, which is the complement of the complementary barcode sequence) is assigned a signal code sequence, and wherein the sample is a cell or tissue sample; b) contacting the sample with a first intermediate probe and a first detectably labeled probe to generate a first complex comprising the first intermediate probe hybridized to the RCA product and the first detectably labeled probe hybridized to the first intermediate probe, wherein the first intermediate probe comprises (i) a hybridization region complementary to the complementary barcode sequence or a portion thereof and (ii) a first overhang sequence, and wherein the first detectably labeled probe comprises (i) a sequence complementary to the first overhang sequence and (ii) a first optically detectable moiety; c) imaging the sample to detect a first signal from the first optically detectable moiety, wherein the first signal corresponds to a first signal code in the signal code sequence; d) contacting the sample with a second intermediate probe and a second detectably labeled probe to generate a second complex comprising the second intermediate probe hybridized to the RCA product and the second detectably labeled probe hybridized to the second intermediate probe, wherein the second intermediate probe comprises (i) a hybridization region complementary to the complementary barcode sequence or a portion thereof and (ii) a second overhang sequence, and wherein the second detectably labeled probe comprises (i) a sequence complementary to the second overhang sequence and (ii) a second optically detectable moiety; and e) imaging the sample to detect a second signal from the second optically detectable moiety, wherein the second signal corresponds to a second signal code in the signal code sequence, wherein the signal code sequence comprising at least the first signal code and the second signal code is determined at a location in the sample, thereby decoding and identifying the variable target sequence at the location in the sample.

In some embodiments, a first RCA product in the sample is generated from a circularized probe formed by ligating an interrogatory probe and a partner probe, where the interrogatory probe comprises the barcode region and targets a variable target sequence in a first target nucleic acid, whereas the partner probe targets a constant target sequence adjacent to the variable target sequence in the first target nucleic acid. In addition to multiple copies of the complementary barcode sequence, the RCA product also comprises multiple copies of the variable target sequence. In some embodiments, the variable target sequence in the RCA product is not detected while the complementary barcode sequence is detected. For instance, in these examples, intermediate probes and detectably labeled probes are provided to decode the complementary barcode sequence (which corresponds to the variant(s) of the first target nucleic acid) but no intermediate probes or detectably labeled probes are used in sequential probe hybridization to decode the variable target sequence.

In some embodiments, a second RCA product in the sample is generated from a circularized probe formed by ligating a circularizable probe provided as a single molecule, where the circularizable probe comprises the barcode region and targets a second target nucleic acid. In addition to multiple copies of the complementary barcode sequence, the RCA product also comprises multiple copies of the target sequence from the second target nucleic acid. In some embodiments, the target sequence (from the second target nucleic acid) in the RCA product is not detected while the complementary barcode sequence is detected. For instance, in these examples, intermediate probes and detectably labeled probes are provided to decode the complementary barcode sequence (which corresponds to the second target nucleic acid) but no intermediate probes or detectably labeled probes are used in sequential probe hybridization to decode the target sequence from the second target nucleic acid.

In some embodiments, the first and second RCA products are generated in the sample, and the sample is contacted with intermediate probes and detectably labeled probes in sequential cycles to decode the complementary barcode sequences in the RCA products, thereby detecting the variant(s) of the first target nucleic acid and detecting the second target nucleic acid using the same decoding scheme. For instance, a first signal code sequence corresponding to a first barcode sequence and the associated variant(s) of the first target nucleic acid and a second signal code sequence corresponding to a second barcode sequence and the associated target nucleic acid (e.g., the second target nucleic acid) can be part of the same codebook.

In some embodiments, the plurality of nucleic acid probes comprises a first intermediate probe comprising i) a first hybridization region which hybridizes to the RCP of the first circularized probe for the first target nucleic acid, and ii) a first detectable region; a first detection oligonucleotide that hybridizes to the first detectable region in the first intermediate probe; a second intermediate probe comprising i) a hybridization region which hybridizes to the RCP of the second circularized probe for the second target nucleic acid, and ii) a second detectable region, and a second detection oligonucleotide that hybridizes to the second detectable region in the second intermediate probe.

In some embodiments, the first hybridization region comprises a sequence in the barcode region corresponding to the variant of the first target nucleic acid, and the second hybridization region comprises a sequence in the barcode region corresponding to the second target nucleic acid. In some embodiments, the first detectable region and the second detectable region are the same or different in sequence. In some embodiments, the first detection oligonucleotide and the second detection oligonucleotide are the same or different in sequence. In some embodiments, the first detection oligonucleotide and the second detection oligonucleotide each comprise the same detectable label or different detectable labels. In some embodiments, in each detection oligonucleotide, the detectable label corresponds to a nucleic acid sequence of the detection oligonucleotide.

In some embodiments, a signal code sequence is assigned to each of i) one or more of the plurality of different variants of the first target nucleic acid and ii) the second target nucleic acid. In some embodiments, a method disclosed herein comprises contacting the biological sample with a Cycle 1 intermediate probe and a Cycle 1 detection oligonucleotide to generate a Cycle 1 complex comprising the Cycle 1 intermediate probe hybridized to one of the RCPs and the Cycle 1 detection oligonucleotide hybridized to the Cycle 1 intermediate probe, wherein the Cycle 1 intermediate probe comprises: i) a Cycle 1 hybridization region which hybridizes to the RCP at a sequence complementary to the barcode region corresponding to the variant(s) of the first target nucleic acid or corresponding to the second target nucleic acid, and ii) a Cycle 1 detectable region, and wherein the Cycle 1 detection oligonucleotide comprises: a sequence complementary to the Cycle 1 detectable region, and a Cycle 1 detectable label. In some embodiments, a method disclosed herein comprises imaging the biological sample to detect a Cycle 1 signal from the Cycle 1 detectable label, wherein the Cycle 1 signal corresponds to a Cycle 1 signal code in the signal code sequence. In some embodiments, a method disclosed herein comprises contacting the biological sample with a Cycle 2 intermediate probe and a Cycle 2 detection oligonucleotide to generate a Cycle 2 complex comprising the Cycle 2 intermediate probe hybridized to the RCP and the Cycle 2 detection oligonucleotide hybridized to the Cycle 2 intermediate probe, wherein the Cycle 2 intermediate probe comprises: i) a Cycle 2 hybridization region which hybridizes to the RCP at the sequence complementary to the barcode region corresponding to the variant(s) of the first target nucleic acid or corresponding to the second target nucleic acid, and ii) a Cycle 2 detectable region, and wherein the Cycle 2 detection oligonucleotide comprises: a sequence complementary to the Cycle 2 detectable region, and a Cycle 2 detectable label. In some embodiments, a method disclosed herein comprises imaging the biological sample to detect a Cycle 2 signal from the Cycle 2 detectable label, wherein the Cycle 2 signal corresponds to a Cycle 2 signal code in the signal code sequence, wherein the signal code sequence comprising at least the Cycle 1 signal code and the Cycle 2 signal code is determined based on signals detected at a location in the biological sample, thereby identifying i) the one or more variants of the first target nucleic acid or ii) the second target nucleic acid at the location in the biological sample.

In some embodiments, the biological sample is contacted with a pool of Cycle 1 intermediate probes and a universal pool of detection oligonucleotides, wherein each different Cycle 1 intermediate probe comprises i) a hybridization region which hybridizes to an RCP corresponding to a different target nucleic acid or a variant thereof, and ii) a detectable region complementary to a detection oligonucleotide of the universal pool of detection oligonucleotides, wherein the biological sample is contacted with a pool of Cycle 2 intermediate probes and the universal pool of detection oligonucleotides, wherein each different Cycle 2 intermediate probe comprises i) a hybridization region which hybridizes to an RCP corresponding to a different target nucleic acid or a variant thereof, and ii) a detectable region complementary to a detection oligonucleotide of the universal pool of detection oligonucleotides.

In some embodiments, a method disclosed herein comprises identifying multiple different subsets of the plurality of different variants of the first target nucleic acid in the biological sample, wherein each subset is assigned a different signal code sequence. In some embodiments, each different subset independently contains one or more variants of different sequences. In some embodiments, a first subset of the variants of the first target nucleic acid contain a wildtype sequence and are assigned a first signal code sequence; a second subset of the variants of the first target nucleic acid contain one, two, three, four, five, or more different mutant sequences and are assigned a second signal code sequence; and the second target nucleic acid is assigned a third signal code sequence.

In some embodiments, a detectably labeled probe hybridizes to a detectable region in an intermediate probe. In some embodiments, the detectable region is in a 5′ overhang and/or a 3′ overhang of the intermediate probe, upon hybridization of the intermediate probe to the barcode region or a portion thereof in an RCP. In some embodiments, the detectable region is a split region, e.g., a portion of the detectable region is in the 5′ overhang and another portion of the detectable region is in the 3′ overhang of an intermediate probe. In some embodiments, the detectable region is in the 5′ overhang of the intermediate probe. In some embodiments, the detectable region is in the 3′ overhang of the intermediate probe. In some embodiments, a first portion of the detectable region is in the 3′ overhang and a second portion of the detectable region is in the 5′ overhang of the intermediate probe.

Provided herein are methods involving the use of one or more probes for analyzing one or more target nucleic acid(s), such as variant sequences in one or more target nucleic acids present in a cell or a biological sample, such as a tissue sample. In some embodiments, the probes include a plurality of intermediate probes and a plurality of detectably labeled probes for combinatorially decoding the hybridization regions of the intermediate probes; since the hybridization regions of the intermediate probes can comprise identifier sequences (e.g., target analyte sequences or barcode sequences), the target nucleic acid in the sample can be identified by decoding the identifier sequences using sequential probe hybridization. In some aspects, the provided embodiments are employed for in situ detection of variant sequences in target nucleic acids in a cell, e.g., in cells of a biological sample or a sample derived from a biological sample, such as a tissue section on a solid support, such as on a transparent slide.

In some aspects, the probes directly or indirectly bind to analytes at locations in the biological sample, and signals associated with the probes are detected at locations in the biological sample to indicate the locations of the analytes. In some embodiments, a plurality of probes are used for sequential hybridization and detection in order to generate a signal code sequence (e.g., a spatiotemporal signal signature) for analytes at each of multiple locations in the biological sample, and the signal code sequence is compared to those in a codebook (e.g., a list of known series of signal codes, also referred to as signal code sequences, wherein the known signal code sequences are assigned to known probe sequences or target analyte sequences) to decode an identifier sequence (e.g., a barcode sequence or an analyte sequence) corresponding to an analyte, thereby identifying analytes at multiple locations in the biological sample. In some embodiments, the signal code sequence comprises signal codes each corresponding to a signal (e.g., signals of different colors correspond to different signal codes) or absence thereof (e.g., dark) at a particular location in the biological sample.

In some aspects, provided herein are in situ assays using microscopy as a readout, e.g., hybridization, or other detection or determination methods involving an optical readout. In some aspects, detection or determination of a sequence of one, two, three, four, five, or more nucleotides of a variable target sequence in a target nucleic acid is performed in situ in a cell in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product or hybridization complex). In some embodiments, a provided method is quantitative and preserves the spatial information within a tissue sample without physically isolating cells or using homogenates. In some embodiments, the present disclosure provides methods for high-throughput profiling of target nucleic acids in situ in a large number of cells, tissues, organs or organisms.

In some aspects, the provided methods comprise imaging the amplification product (e.g., RCA product) via binding of an intermediate probe and a detectably labeled probe (e.g., a detection oligonucleotide comprising a fluorescent label), and detecting the detectable label. In some embodiments, a signal associated with a detectably labeled oligonucleotide is measured and quantitated. The terms “label” and “detectable label” comprise a directly or indirectly detectable moiety that is associated with (e.g., conjugated to) a molecule to be detected, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. In some embodiments, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

In some embodiments, an RCP or a probe disclosed herein comprises one or more detectably labelled, e.g., fluorescent, nucleotides. In some embodiments, the one or more detectably labelled nucleotides are incorporated during generation of the RCP (e.g., during RCA) or the probe. Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). For exemplary methods for custom synthesis of nucleotides having other fluorophores, see, Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of an RCP, an intermediate probe, and/or a detectably labeled oligonucleotide bound to the RCP or to the intermediate probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of an RCP, an intermediate probe, and/or a detectably labeled oligonucleotide bound to the RCP or to the intermediate probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

### VI. Samples and Sample Processing

A sample disclosed herein can be derived from any biological sample. The sample may not be limited to any specific source, but may be peripheral blood mononuclear cells, tumors, tissue, bone marrow, biopsies, serum, blood, plasma, saliva, lymph fluid, pleura fluid, cerebrospinal and synovial fluid. The sample may be extracted from a subject. Samples extracted from individuals may be subjected to the methods described herein to identify and evaluate immune responses during cancer and disease or subsequent to immunotherapy.

Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components.

Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, the biological sample corresponds to cells (e.g., derived from a cell culture, a tissue sample, or cells deposited on a surface). In a cell sample with a plurality of cells, individual cells can be naturally unaggregated. For example, the cells can be derived from a suspension of cells (e.g., a body fluid such as blood) and/or disassociated or disaggregated cells from a tissue or tissue section. The number of cells in the biological sample can vary. Some biological samples comprise large numbers of cells, e.g., blood samples, while other biological samples comprise smaller or only a small number of cells or may only be suspected of containing cells, e.g., plasma, serum, urine, saliva, synovial fluids, amniotic fluid, lachrymal fluid, lymphatic fluid, liquor, cerebrospinal fluid and the like.

In some embodiments, a cell-containing biological sample comprises a body fluid or a cell-containing sample derived from the body fluid, e.g., whole blood, samples derived from blood such as plasma or serum, buffy coat, urine, sputum, lachrymal fluid, lymphatic fluid, sweat, liquor, cerebrospinal fluid, ascites, milk, stool, bronchial lavage, saliva, amniotic fluid, nasal secretions, vaginal secretions, semen/seminal fluid, wound secretions, cell culture and swab samples, or any cell-containing sample derived from the aforementioned samples. In some embodiments, a cell-containing biological sample can be a body fluid, a body secretion or body excretion, e.g., lymphatic fluid, blood, buffy coat, plasma or serum. In some embodiments, a cell-containing biological sample can be a circulating body fluid such as blood or lymphatic fluid, e.g., peripheral blood obtained from a mammal such as human.

The biological sample can include any number of macromolecules, for example, cellular macromolecules and organelles (e.g., mitochondria and nuclei). In some embodiments, the biological sample is obtained as a tissue sample, such as a tissue section, biopsy, a core biopsy, needle aspirate, or fine needle aspirate. In some embodiments, the biological sample is from a cell block or a cell pellet. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface. In some embodiments, the biological sample comprises transcripts of antigen receptor molecules.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate is coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample is prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples are destained. Any suitable methods of any suitable destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) is isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto are anchored to a polymer matrix. For example, the polymer matrix can be a hydrogel. In some embodiments, one or more of the polynucleotide probe(s) and/or amplification product (e.g., amplicon) thereof is/are modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample is permeabilized to facilitate transfer of species (such as probes) into the sample. If a sample is not permeabilized sufficiently, the amount of species (such as probes) in the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample is permeabilized by non-chemical permeabilization methods. Non-chemical permeabilization methods can be used. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(ix) Selective Enrichment of RNA or cDNA Species

In some embodiments, where RNA or cDNA is the analyte, one or more RNA or cDNA analyte species of interest is selectively enriched. For example, one or more species of RNA or cDNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs or cDNAs of interest can be used to amplify the one or more RNAs or cDNAs of interest, thereby selectively enriching these RNAs or cDNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of suitable methods (e.g., streptavidin beads).

In some embodiments, the analytes may be further enriched for in situ readout by immobilization at a location in the biological sample. In a non-limiting example, the analytes may comprise one or more fragments that are specific to a location in the biological sample.

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

### VII. Compositions, Kits, and Systems

Provided herein are kits, for example comprising one or more oligonucleotides, e.g., circularizable probes, partner probe, interrogatory probe, etc. as described in Sections I-V, and instructions for performing the methods provided herein. For example, provided herein is a kit for analyzing a biological sample, comprising a partner probe and a plurality of interrogatory probes, wherein the partner probe comprises i) a constant hybridization region complementary to a constant target sequence in a target nucleic acid, and ii) an overhang, wherein each interrogatory probe comprises i) an interrogatory hybridization region complementary to a variant among a plurality of different variants of a variable target sequence in the target nucleic acid, wherein the interrogatory hybridization region comprises one or more internal interrogatory nucleotides, and ii) a barcode region corresponding to the variant, and wherein two or more different interrogatory probes of the plurality of interrogatory probes are configured to compete for hybridization to a particular variant of the variable target sequence in a molecule of the target nucleic acid. In some embodiments, the kit comprises one or more reagents for probe circularization, one or more reagents for rolling circle amplification (RCA) of a circularized probe, and/or one or more reagents for detecting an RCA product (RCP). In some embodiments, provided herein is a library of interrogatory probes comprising each comprising a sequence in its interrogatory hybridization region that is complementary to a nucleotide variation, a nucleotide polymorphism, a mutation, a substitution, an insertion, a deletion, a translocation, a duplication, an inversion, and/or a repetitive sequence. In some embodiments, the kit comprises a library of circularizable probes for a panel of analytes (e.g., panel of gene transcripts).

In some embodiments, the kits further comprise one or more reagents for performing the methods provided herein. In some embodiments, the kits further comprise one or more reagents required for one or more steps comprising hybridization, ligation, extension, amplification, detection, and/or sample preparation as described herein. In some embodiments, the kit further comprises any one or more of the intermediate probes and detectably labeled oligonucleotides disclosed herein, e.g., as described in Sections V. In some embodiments, any or all of the oligonucleotides are DNA molecules.

In some embodiments, the kit further comprises an enzyme such as a ligase and/or a polymerase described herein. In some embodiments, the ligase has DNA-splinted DNA ligase activity. In some embodiments, the kit comprises a polymerase, for instance for performing extension of the primers to incorporate modified nucleotides into cDNA products of antigen receptor transcripts. In some embodiments, the kits may contain reagents for forming a functionalized matrix (e.g., a hydrogel), such as any suitable functional moieties. In some examples, also provided are buffers and reagents for tethering the modified primers, cDNA products, and/or RCA products to the functionalized matrix. The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as detectably labeled oligonucleotides or detectable labels. In some embodiments, the kits optionally contain other components, for example nucleic acid primers, enzymes and reagents, buffers, nucleotides, modified nucleotides, reagents for additional assays.

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., as described in Section V). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules (e.g., as described in Section II) in their naturally occurring place (e.g., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

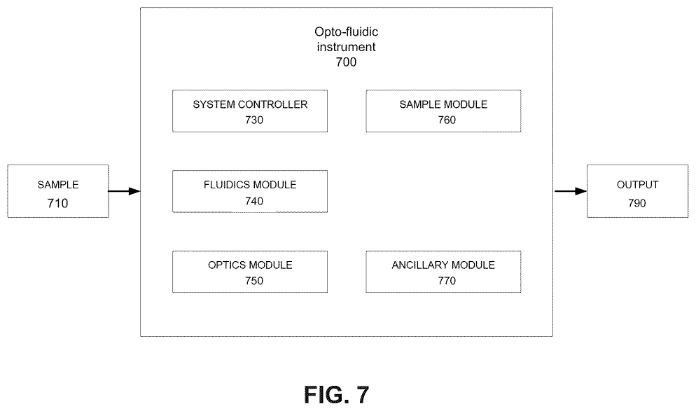

FIG. 7 shows an example workflow of analysis of a biological sample 710 (e.g., cell or tissue sample) using an opto-fluidic instrument 700, according to various embodiments. In various embodiments, the sample 710 can be a biological sample (e.g., a tissue) that includes molecules such as DNA, RNA, proteins, antibodies, etc. For example, the sample 710 can be a sectioned tissue that is treated to access the RNA thereof for labeling with probes described herein (e.g., in Section III). Ligation of the probes may generate a circular probe which can be enzymatically amplified and bound with detectably labeled probes, which can create bright signal that is convenient to image and has a high signal-to-noise ratio.

In various embodiments, the sample 710 may be placed in the opto-fluidic instrument 700 for analysis and detection of the molecules in the sample 710. In various embodiments, the opto-fluidic instrument 700 can be a system configured to facilitate the experimental conditions conducive for the detection of the target molecules. For example, the opto-fluidic instrument 700 can include a fluidics module 740, an optics module 750, a sample module 760, and an ancillary module 770, and these modules may be operated by a system controller 730 to create the experimental conditions for the probing of the molecules in the sample 710 by selected probes (e.g., circularizable DNA probes, partner probes and interrogatory probes), as well as to facilitate the imaging of the probed sample (e.g., by an imaging system of the optics module 750). In various embodiments, the various modules of the opto-fluidic instrument 700 may be separate components in communication with each other, or at least some of them may be integrated together.

In various embodiments, the sample module 760 may be configured to receive the sample 710 into the opto-fluidic instrument 700. For instance, the sample module 760 may include a sample interface module (SIM) that is configured to receive a sample device (e.g., cassette) onto which the sample 710 can be deposited. That is, the sample 710 may be placed in the opto-fluidic instrument 700 by depositing the sample 710 (e.g., the sectioned tissue) on a sample device that is then inserted into the SIM of the sample module 760. In some instances, the sample module 760 may also include an X-Y stage onto which the SIM is mounted. The X-Y stage may be configured to move the SIM mounted thereon (e.g., and as such the sample device containing the sample 710 inserted therein) in perpendicular directions along the two-dimensional (2D) plane of the opto-fluidic instrument 700. Additional discussion related the SIM can be found in U.S. Provisional Application No. 63/348,879, filed Jun. 3, 2022, titled “Methods, Systems, and Devices for Sample Interface,” which is incorporated herein by reference in its entirety.

The experimental conditions that are conducive for the detection of the molecules in the sample 710 may depend on the target molecule detection technique that is employed by the opto-fluidic instrument 700. For example, in various embodiments, the opto-fluidic instrument 700 can be a system that is configured to detect molecules in the sample 710 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 710 using reagents such as washing/stripping reagents, hybridizing reagents, etc., and such reagents may be provided by the fluidics module 740.

In various embodiments, the fluidics module 740 may include one or more components that may be used for storing the reagents, as well as for transporting said reagents to and from the sample device containing the sample 710. For example, the fluidics module 740 may include reservoirs configured to store the reagents, as well as a waste container configured for collecting the reagents (e.g., and other waste) after use by the opto-fluidic instrument 700 to analyze and detect the molecules of the sample 710. Further, the fluidics module 740 may also include pumps, tubes, pipettes, etc., that are configured to facilitate the transport of the reagent to the sample device (e.g., and as such the sample 710). For instance, the fluidics module 740 may include pumps (“reagent pumps”) that are configured to pump washing/stripping reagents to the sample device for use in washing/stripping the sample 710 (e.g., as well as other washing functions such as washing an objective lens of the imaging system of the optics module 750).

In various embodiments, the ancillary module 770 can be a cooling system of the opto-fluidic instrument 700, and the cooling system may include a network of coolant-carrying tubes that are configured to transport coolants to various modules of the opto-fluidic instrument 700 for regulating the temperatures thereof. In such cases, the fluidics module 740 may include coolant reservoirs for storing the coolants and pumps (e.g., “coolant pumps”) for generating a pressure differential, thereby forcing the coolants to flow from the reservoirs to the various modules of the opto-fluidic instrument 700 via the coolant-carrying tubes. In some instances, the fluidics module 740 may include returning coolant reservoirs that may be configured to receive and store returning coolants, e.g., heated coolants flowing back into the returning coolant reservoirs after absorbing heat discharged by the various modules of the opto-fluidic instrument 700. In such cases, the fluidics module 740 may also include cooling fans that are configured to force air (e.g., cool and/or ambient air) into the returning coolant reservoirs to cool the heated coolants stored therein. In some instance, the fluidics module 740 may also include cooling fans that are configured to force air directly into a component of the opto-fluidic instrument 700 so as to cool said component. For example, the fluidics module 740 may include cooling fans that are configured to direct cool or ambient air into the system controller 730 to cool the same.

As discussed above, the opto-fluidic instrument 700 may include an optics module 750 which include the various optical components of the opto-fluidic instrument 700, such as but not limited to a camera, an illumination module (e.g., LEDs), an objective lens, and/or the like. The optics module 750 may include a fluorescence imaging system that is configured to image the fluorescence emitted by the probes (e.g., oligonucleotides) in the sample 710 after the probes are excited by light from the illumination module of the optics module 750.

In some instances, the optics module 750 may also include an optical frame onto which the camera, the illumination module, and/or the X-Y stage of the sample module 760 may be mounted.

In various embodiments, the system controller 730 may be configured to control the operations of the opto-fluidic instrument 700 (e.g., and the operations of one or more modules thereof). In some instances, the system controller 730 may take various forms, including a processor, a single computer (or computer system), or multiple computers in communication with each other. In various embodiments, the system controller 730 may be communicatively coupled with data storage, set of input devices, display system, or a combination thereof. In some cases, some or all of these components may be considered to be part of or otherwise integrated with the system controller 730, may be separate components in communication with each other, or may be integrated together. In other examples, the system controller 730 can be, or may be in communication with, a cloud computing platform.

In various embodiments, the opto-fluidic instrument 700 may analyze the sample 710 and may generate the output 790 that includes indications of the presence of the target molecules in the sample 710. For instance, with respect to the example embodiment discussed above where the opto-fluidic instrument 700 employs a hybridization technique for detecting molecules, the opto-fluidic instrument 700 may cause the sample 710 to undergo successive rounds of detectably labeled probe hybridization (e.g., using two or more sets of fluorescent probes, where each set of fluorescent probes is excited by a different color channel) and be imaged to detect target molecules in the probed sample 710. In such cases, the output 790 may include optical signatures (e.g., a codeword) specific to each gene, which allow the identification of the target molecules.

### VIII. Applications

In some aspects, the provided embodiments can be applied in an in situ method of analyzing nucleic acid sequences in intact tissues or samples in which the spatial information has been preserved. In some aspects, the embodiments can be applied in an imaging or detection method for multiplexed nucleic acid analysis. In some aspects, the provided embodiments can be used to identify or detect mutations in a target nucleic acid. In some aspects, the provided embodiments can be used to crosslink the RCA products via modified nucleotides, e.g., to a matrix, to increase the stability of the RCA products in situ.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples. In some aspects, the embodiments can be applied to visualize the distribution of genetically encoded markers in whole tissue at subcellular resolution.

### IX. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some embodiments, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The examples are included for illustrative purposes only and are not intended to limit the scope of the present disclosure.

### Example 1: Detecting KRAS Mutation In Situ Using Probe Pairs

This example demonstrates that probe pairs comprising partner probes and interrogatory probes can be used for KRAS mutation detection in situ.

ME-180 cells primarily comprise wildtype KRAS, and A549 cells primarily comprise a G to A mutation in KRAS. Cells were mounted on glass slides, fixed by incubating in paraformaldehyde (PFA), washed, and permeabilized using Triton-X and washed.

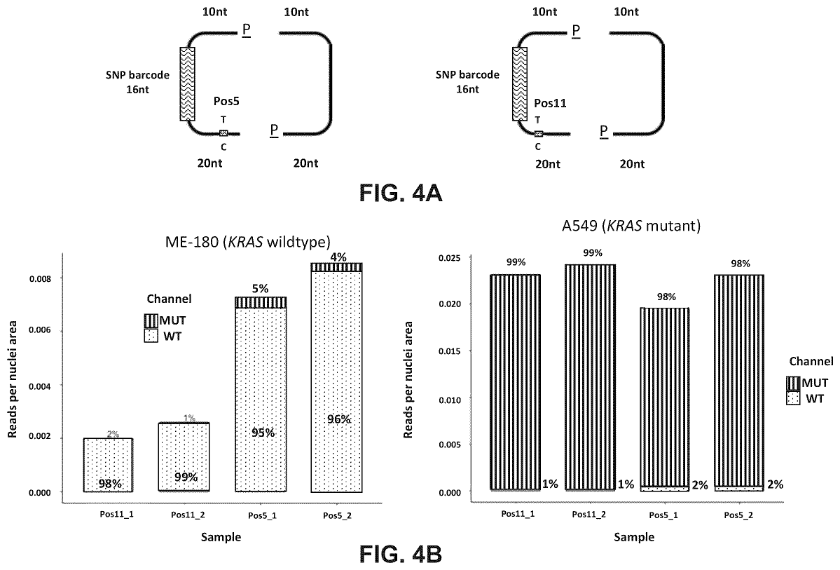

As shown in FIG. 4A, two probe pairs were designed. Probe Pair “Pos5” included an interrogatory probe (comprising an SNP barcode) and a partner probe (shown with an optional gene barcode), where the SNP interrogatory probe had an interrogatory nucleotide on the arm targeting the wildtype or the mutant KRAS allele, at position 5 from the ligation junction with the partner probe. Probe Pair “Pos11” included an interrogatory probe (comprising an SNP barcode) and a partner probe (shown with an optional gene barcode), where the SNP interrogatory probe had an interrogatory nucleotide on the arm targeting the wildtype or the mutant KRAS allele, at position 11 from the ligation junction with the partner probe. The interrogatory nucleotide C is shown to target the wildtype G and the interrogatory nucleotide T is shown to target the mutant A.

The probes were applied to the cells and allowed to hybridize, after which the cells were washed (e.g., using a buffer compatible with the following ligation reaction). The sample was then incubated with a ligase in a ligation buffer to form circularized probes. Two ligations were performed: (1) to ligate the splint hybridization regions of the partner probe and the interrogatory probe; (2) to ligate the constant hybridization region of the partner probe to the interrogatory hybridization region of the interrogatory probe. For RCA, the cells were washed and then incubated in an RCA reaction mixture (containing Phi29 reaction buffer, dNTPs, Phi29 polymerase) to generate RCA products (RCPs) corresponding to wildtype or mutant KRAS.

Detection of the barcode sequences was performed using intermediate probes that hybridize to the RCPs and detectably labeled detection oligonucleotides (DOs) that hybridize to the overhangs of the intermediate probes. The probes were hybridized to the RCPs in situ in a hybridization buffer. The cells were washed, stained with DAPI, and mounted in a mounting medium for imaging using fluorescent microscopy, and RCPs counts per unit nuclei area were detected.

FIG. 4B shows RCPs per nuclei area detected in ME-180 cells (left panel) and A549 cells (right panel) using Probe Pair “Pos5” and Probe Pair “Pos11.” In ME-180 cells (primarily KRAS wildtype), both probe pairs detected higher frequencies of wildtype than mutant KRAS RCPs. In A549 cells (primarily KRAS mutant), both probe pairs detected higher frequencies of mutant than wildtype KRAS RCPs. These results show that detection of KRAS single nucleotide variations using probe pairs having internal interrogatory nucleotides provided good discrimination between nucleotide variations. In another experiment (results not shown), a probe pair comprising a partner probe with a short spacer region (5 nucleotides) showed similar results for detection of variant sequences.

### Example 2: Detecting KRAS Mutation In Situ Using Probe Pairs Combined with Circularizable Probes

This example demonstrates that probe pairs comprising partner probes and interrogatory probes can be used for KRAS mutation detection in situ, and the detection was compatible with RCA-based detection using circularizable probes provided as one oligonucleotide.

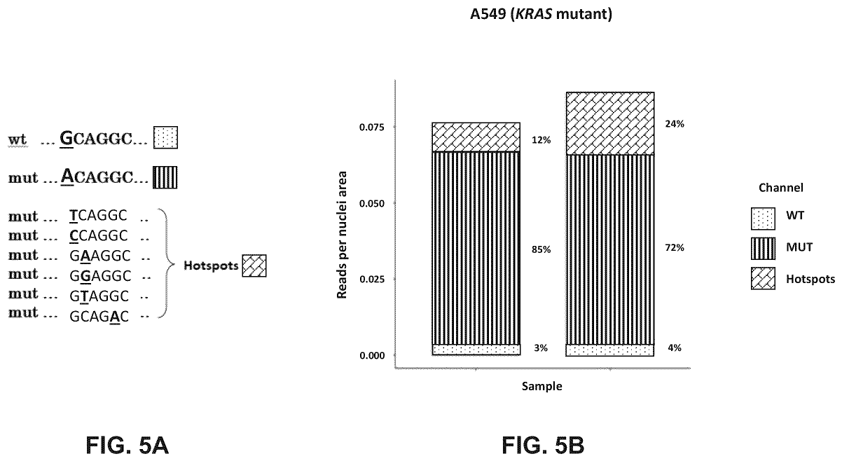

Probe pairs were designed essentially as described in Example 1. As shown in FIG. 5A, three probe pairs were used: a first probe pair for detecting the wildtype G in KRAS, where the interrogatory probe in the pair contained a barcode sequence corresponding to the wildtype G; a second probe pair for detecting the mutant A in KRAS, where the interrogatory probe in the pair contained a barcode sequence corresponding to the mutant A; and a third probe “pair” for detecting multiple other mutations (“hotspots”) in KRAS, where the probe “pair” contained a common partner probe and multiple different interrogatory probes each targeting a different hotspot mutation but sharing the same barcode sequence corresponding to the hotspots. In other words, the third probe “pair” is a probe set comprising a common partner probe targeting a constant target sequence and multiple interrogatory probes targeting nucleotide(s) of interest in a variable target sequence.

The probe pairs for KRAS detection were combined with circularizable probes (e.g., padlock probes) having symmetric arms that were each 20 nucleotides in length. Probe hybridization, ligation, RCA, and barcode signal detection were performed essentially as described in Example 1, and the probe pairs for KRAS and the symmetric padlock probes were subjected to the same workflow and conditions. FIG. 5B shows RCPs per nuclei area detected in A549 cells (primarily KRAS mutant). RCPs corresponding to the wildtype, mutant A, as well as the hotspot mutations in KRAS were detected. The detection specificity of KRAS mutations using the probe pairs were maintained when combined with the symmetric padlock probes under the same conditions for probe hybridization, ligation, RCA, and barcode signal detection.

These results show that probe pairs comprising two oligonucleotides (e.g., partner probes and interrogatory probes) provided good discrimination between nucleotide variations and the assay using these probe pairs was compatible with RCA-based detection using circularizable probes provided as one oligonucleotide.

### Example 3: Detecting Mutations In Situ Using a Panel of Probe Pairs Combined with a Panel of Circularizable Probes

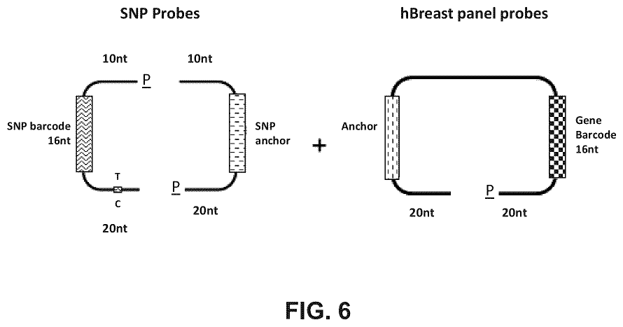

Probe pairs comprising partner probes and interrogatory probes were designed for detecting a panel of genes each having a wildtype and a mutant allele, essentially as described in Example 1 and Example 2. The panel of probe pairs was used in combination with circularizable probes each provided as one oligonucleotide for detecting a panel of gene transcripts in FFPE human breast (hBreast) tissue. FIG. 6 depicts the SNP probes used to detect the wildtype/mutant pairs for each gene in the SNP panel (only probes for the KRAS wildtype or mutant c.38G>A alleles are shown for simplicity), and hBreast panel probes (by way of example, a schematic of a circularizable probe is shown).

Probe hybridization, ligation, RCA, and barcode signal detection were performed essentially as described in Example 1 and Example 2, and the probe pairs for the SNP panel (e.g., comprising wildtype and mutant for each gene of a plurality of genes) and the probes for the hBreast panel were subjected to the same workflow and conditions. Allele frequency of wildtype and mutant genes were detected using bulk RNA sequencing of the entire section. RCPs detected in the wildtype and mutant tissue samples, and RCP counts were determined corresponding to wildtype and mutant alleles of the probed genes. It was observed that in situ allele detection using a panel of probe pairs are compatible with a panel of circularizable probes each provided as single molecules for use in RCA-based detection in tissue samples. Using different probe designs that required additional ligation steps as compared to an assay using only a single probe type, the observations support the use of a combination of probe types for detecting multiple analytes that may require different levels of specificity and/or stringency for detection (e.g., SNPs and a panel of gene transcripts). In addition, in situ allele detection of a panel of genes can be correlated with allele frequencies of the genes detected using bulk RNA sequencing of the tissue samples.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1-116. (canceled)

117. A method for analyzing a biological sample, comprising:
a) contacting the biological sample with:
i) a partner probe and an interrogatory probe for a first target nucleic acid,
wherein the partner probe comprises i) a constant hybridization region complementary to a constant target sequence in the first target nucleic acid, and ii) an overhang,
wherein the interrogatory probe comprises i) an interrogatory hybridization region complementary to a variant among a plurality of different variants of a variable target sequence in the first target nucleic acid, and ii) a barcode region corresponding to the variant, and
ii) a circularizable probe for a second target nucleic acid, comprising i) a hybridization region complementary to a target sequence in the second target nucleic acid, and ii) a barcode region corresponding to the second target nucleic acid, wherein the circularizable probe is a single molecule;
b) ligating the partner probe and the interrogatory probe hybridized to the first target nucleic acid, thereby generating a first circularized probe comprising the barcode region corresponding to the variant of the first target nucleic acid, and
ligating the circularizable probe hybridized to the second target nucleic acid, thereby generating a second circularized probe comprising the barcode region corresponding to the second target nucleic acid;
c) generating a rolling circle amplification product (RCP) of each of the first and second circularized probes; and
d) detecting signals associated with the complements of the barcode regions in the RCPs at locations in the biological sample, thereby detecting the variant of the first target nucleic acid and detecting the second target nucleic acid at the locations in the biological sample.

118. The method of claim 117, wherein the interrogatory hybridization region comprises one or more internal interrogatory nucleotides, and each internal interrogatory nucleotide is complementary to a corresponding nucleotide of interest in the variant.

119. The method of claim 118, wherein the nucleotide at a free 5′ terminus of the interrogatory hybridization region is at position 1, and each internal interrogatory nucleotide is at a nucleotide position between position 3 and position 10, inclusive, in the interrogatory hybridization region.

120. The method of claim 117, wherein the interrogatory hybridization region comprises a single internal interrogatory nucleotide complementary to a corresponding single nucleotide of interest in the variant, and the single nucleotide of interest is a single nucleotide variation (SNV), a single nucleotide polymorphism (SNP), a point mutation, a single nucleotide substitution, a single nucleotide insertion, or a single nucleotide deletion.

121. The method of claim 117, wherein the constant hybridization region in the partner probe or the interrogatory hybridization region in the interrogatory probe is independently of between 5 and 50 nucleotides in length.

122. The method of claim 117, wherein the constant hybridization region in the partner probe is longer than the interrogatory hybridization region in the interrogatory probe.

123. The method of claim 117, wherein the barcode region in the interrogatory probe comprises a barcode sequence specific to the variant, and the partner probe comprises a barcode sequence specific to the first target nucleic acid but not specific to any one or more variants of the variable target sequence in the first target nucleic acid.

124. The method of claim 117, wherein the overhang in the partner probe comprises a spacer region that is common among partner probes for two or more different target nucleic acids.

125. The method of claim 117, wherein the partner probe and the interrogatory probe each comprises a splint hybridization region complementary to a splint oligonucleotide, and upon hybridization to the splint oligonucleotide, the splint hybridization regions are configured to be ligated using the splint oligonucleotide as a template, with or without gap filling and/or cleavage of a 5′ flap prior to the ligation.

126. The method of claim 125, wherein the splint hybridization region in the interrogatory probe and/or the splint hybridization region in the partner probe comprises a barcode region corresponding to the first target nucleic acid or a sequence thereof.

127. The method of claim 125, wherein the barcode region corresponding to the variant is in the splint hybridization region in the interrogatory probe.

128. The method of claim 117, wherein the circularizable probe for the second target nucleic acid comprises i) a single hybridization region complementary to the target sequence in the second target nucleic acid, and ii) 5′ and 3′ splint hybridization regions complementary to a splint oligonucleotide, and upon hybridization to the splint oligonucleotide, the 5′ and 3′ splint hybridization regions are configured to be ligated using the splint oligonucleotide as a template, with or without gap filling and/or cleavage of a 5′ flap prior to the ligation.

129. The method of claim 117, wherein the circularizable probe for the second target nucleic acid comprises a split hybridization region.

130. The method of claim 117, wherein the first target nucleic acid and the second target nucleic acid are RNA transcripts of different genes.

131. The method of claim 117, wherein the biological sample is contacted with a plurality of different interrogatory probes, wherein each different interrogatory probe comprises i) an interrogatory hybridization region complementary to a different variant among the plurality of different variants of the variable target sequence in the first target nucleic acid, and ii) a barcode region corresponding to the different variant.

132. The method of claim 131, wherein the plurality of different variants comprise nucleotides of interest at two or more nucleotide positions in the variable target sequence.

133. The method of claim 131, comprising washing the biological sample after contacting with the plurality of different interrogatory probes.

134. The method of claim 117, comprising contacting the biological sample with a plurality of nucleic acid probes, wherein each nucleic acid probe:
i) comprises a hybridization region complementary to a sequence in one of the RCPs; and
ii) is detectably labeled or comprises a detectable region that directly or indirectly binds to a detection oligonucleotide comprising a detectable label.

135. The method of claim 117, wherein the biological sample is a cell or tissue sample.

136. The method of claim 117, wherein the detecting in step (d) comprises sequencing the complements of the barcode regions in the RCPs.
